(19) United States
(12) Patent Application Publication
(10) Pub. No.: US 2022/0331395 A1
(43) Pub. Date: Oct. 20, 2022

(54) EXTENDED-RELEASE INJECTABLE GEL FORMULATIONS CONTAINING ANGIOTENSIN-(1-7) OLIGOPEPTIDES OR VARIANTS THEREOF

(71) Applicant: ProNeurogen, Inc., Tucson, AZ (US)

(72) Inventors: Meredith HAY, Tucson, AZ (US); Nina OSSANNA, Tucson, AZ (US); Christopher A. RHODES, San Diego, CA (US); Lawrence J. D'SOUZA, San Diego, CA (US); Katrina PENTICOFF, San Diego, CA (US)

(21) Appl. No.: 17/715,724

(22) Filed: Apr. 7, 2022

Related U.S. Application Data
(60) Provisional application No. 63/171,890, filed on Apr. 7, 2021.

(51) Int. Cl. A61K 38/08; A61K 9/00; A61K 47/34; A61P 25/28; A61K 47/12

(52) U.S. Cl. CPC A61K 38/085; A61K 9/0024; A61K 47/34; A61P 25/28; A61K 47/12

(57) ABSTRACT

The present disclosure provides an extended-release gel formulation containing a biocompatible polymer and an angiotensin-(1-7) oligopeptide or a variant thereof. Also provided are methods of treating subjects with vascular dementia, e.g., using the formulations disclosed herein or compositions containing the same, a subject can be administered the extended-release gel formulation to treat the vascular dementia.

### SEQUENCE LISTING

The instant application contains a Sequence Listing which has been submitted electronically in ASCII format and is hereby incorporated by reference in its entirety. Said ASCII copy, created on Apr. 6, 2022, is named 51568-002002_Sequence_Listing_4_7_22_ST25 and is 9,110 bytes in size.

### FIELD OF THE DISCLOSURE

The present disclosure relates to extended-release gel formulations containing angiotensin-(1-7) oligopeptides or variants thereof and a biocompatible polymer which can be used for the treatment of vascular dementia in a subject.

### BACKGROUND

Biodegradable polymers are often leveraged for their ability to be configured into effective controlled-release drug vehicles. In particular, polymer matrices synthesized from poly-(lactic acid)(PLA) or poly-(lactic co-glycolic acid)(PLGA) chains have been demonstrated to be particularly useful due to their biodegradability and tolerability and have been employed in a number of commercially-available drugs approved by the U.S. Food and Drug Administration (e.g., LUPRON DEPOT®). However, a major problem with PLA/PLGA-based drug delivery systems is their tendency to release large amounts of therapeutic cargo during an initial burst release phase which occurs at the time of administration and shortly thereafter. The initial release problem is particularly challenging with highly water-soluble drugs. This problematic release property may result in adverse effects resulting from excessive drug release and may preclude sustained availability of the drug throughout the treatment window, thereby requiring more frequent administration of the drug, which may result in poorer patient compliance and greater patient discomfort.

Therefore, there is an urgent need for improved extended-release formulations that reduce or eliminate the initial burst release of therapeutic cargo and provide an extended-release profile when administered to a subject.

### SUMMARY OF THE DISCLOSURE

The present disclosure relates to compositions containing an extended-release injectable gel formulation containing an angiotensin-(1-7) oligopeptide (ANG-(1-7)) or a variant thereof and a biocompatible polymer. Accordingly, the present disclosure provides pharmaceutical compositions containing the disclosed formulation that can be administered to a subject (e.g., a human), such as a human subject having or at risk of developing a cognitive impairment (e.g., vascular dementia), thereby treating the cognitive impairment.

In a first aspect, the disclosure provides an extended-release injectable gel formulation including at least one biocompatible polymer including polylactic acid (PLA) or poly(lactic-co-glycolic acid) (PLGA) having dispersed therein an effective amount of an oligopeptide of SEQ ID NO: 1, wherein the polymer and the oligopeptide are dissolved in an organic solvent, wherein the formulation is configured to have an in vitro release profile including a sustained release of at least 60% (e.g., at least 65%, 70%, 75%, 80%, 85%, 90%, 91%, 92%, 93%, 94%, 95%, 96%, 97%, 98%, 99% or more) of the effective amount of the oligopeptide within 48 hours following placement of the formulation in a release medium (to) and an initial burst release not greater than 30% (e.g., not greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, or 29%) of the effective amount of the oligopeptide within 24 hours following t₀, wherein the sustained release does not exceed an average rate of release of the oligopeptide that is greater than 20%/24 hours for a period equal to or greater than seven days following t₀, as measured by high performance liquid chromatography (HPLC) and/or mass spectrometry (MS) at an operating temperature of 37° C., wherein the release medium is phosphate buffered saline (PBS) having a temperature of 37° C. and pH 7.4.

In another aspect, the disclosure provides an extended-release injectable gel formulation including at least one biocompatible polymer including PLA or PLGA having dispersed therein an effective amount of an oligopeptide of SEQ ID NO: 1, wherein the polymer and the oligopeptide are dissolved in an organic solvent, wherein the formulation is configured to have an in vitro release profile including a sustained release of at least 60% (e.g., at least 65%, 70%, 75%, 80%, 85%, 90%, 91%, 92%, 93%, 94%, 95%, 96%, 97%, 98%, 99% or more) of the effective amount the oligopeptide within 25 days following placement of the formulation in a release medium (t₀) and an initial burst release not greater than 30% (e.g., not greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, or 29%) of the effective amount of the oligopeptide within 24 hours following t₀, wherein the sustained release does not exceed an average rate of release of the oligopeptide that is greater than 30%/168 hours for a period equal to or greater than 30 days following t₀, as measured by HPLC and/or MS at an operating temperature of 37° C., wherein the release medium is PBS having a temperature of 37° C. and pH 7.4.

In another aspect, the disclosure provides an extended-release injectable gel formulation including at least one biocompatible polymer including PLA or PLGA and having dispersed therein an effective amount of an oligopeptide of SEQ ID NO: 1, wherein the polymer and the oligopeptide are dissolved in an organic solvent, wherein, following subcutaneous or intramuscular administration to a human subject, the formulation is configured to form a depot in vivo that releases the oligopeptide at a rate sufficient to maintain an average serum concentration of between 1-1000 ng/mL (e.g., 2-900, 5-800, 10-700, 20-600, 30-500, 40-400, 50-300, 60-200, 70-100, or 80-90 ng/mL) for a period of 24-168 hours (e.g., 25-167, 30-126, 36-84, or 42-60 hours) following administration, and a maximum serum concentration (Cₘₐₓ) of the oligopeptide of between 1-1000 ng/mL (e.g., 2-900, 5-800, 10-700, 20-600, 30-500, 40-400, 50-300, 60-200, 70-100, or 80-90 ng/mL) for a period of 48 hours following administration.

In another aspect, the disclosure provides an extended-release injectable gel formulation including at least one biocompatible polymer including PLA or PLGA and having dispersed therein an effective amount of an oligopeptide of SEQ ID NO: 1, wherein the polymer and the oligopeptide are dissolved in an organic solvent, wherein, following subcutaneous or intramuscular administration to a human subject, the formulation is configured to form a depot in vivo that releases the oligopeptide at a rate sufficient to maintain an average serum concentration of between 1 and 1000 ng/mL (e.g., 2-900, 5-800, 10-700, 20-600, 30-500, 40-400, 50-300, 60-200, 70-100, or 80-90 ng/mL) for a period of 21 days following administration, a Cₘₐₓ of the oligopeptide of between 1 and 1000 ng/mL (e.g., 2-900, 5-800, 10-700, 20-600, 30-500, 40-400, 50-300, 60-200, 70-100, or 80-90 ng/mL) for a period of 21 days following administration.

In some embodiments of any of the above aspects, the formulation includes PLA in an amount of 100% of a total number of monomers in the biocompatible polymer and the biocompatible polymer has a molecular weight of between 10,000 and 18,000 Daltons (e.g., 11,000-17,000; 12,000-16,000; 13,000-15,000; or 13,500-14,500 Daltons).

In some embodiments of any of the above aspects, the PLGA includes PLA in an amount of 75% and polyglycolic acid (PGA) in an amount of 25% of a total number of monomers in the biocompatible polymer and the biocompatible polymer has a molecular weight of between 4,000 and 15,000 Daltons (e.g., 5,000-14,000; 6,000-13,000; 7,000-12,000; 8,000-11,000; or 9,000-10,000 Daltons).

In some embodiments of any of the above aspects, the PLGA includes PLA in an amount of 50% and PGA in an amount of 50% of a total number of monomers in the biocompatible polymer and the biocompatible polymer has a molecular weight of between 7,000 and 17,000 Daltons (e.g., 8,000-16,000; 9,000-15,000; 10,000-14,000; 11,000-13,000; or 12,000-12,500 Daltons).

In some embodiments, the PLGA is ester-capped or acid-capped. In some embodiments, the PLA is ester-capped or acid-capped.

In some embodiments of any of the above aspects, the formulation includes the biocompatible polymer in an amount of 1-300 mg (e.g., 2-290, 5-280, 10-270, 20-260, 30-250, 40-240, 50-230, 60-220, 70-210, 80-200, 90-190, 100-180, 110-170, 120-160, 130-150, or 140-145 mg). In some embodiments, the biocompatible polymer includes 1-50% (w/w)(e.g., 2-45%, 5-40%, 10-35%, 15-30%, or 20-25% (w/w)) of a total mass of the formulation.

In some embodiments of any of the above aspects, the organic solvent is dimethyl sulfoxide (DMSO), benzoic acid (BzOH), N-methyl-2-pyrrolidone (NMP), benzyl benzoate (BB) or any combination thereof. In some embodiments, the solvent is NMP. In some embodiments, the combination includes DMSO and BzOH or DMSO and BB.

In some embodiments of any of the above aspects, the formulation further includes a release modifier. In some embodiments, the release modifier is selected from the group consisting of hydrophobic carboxylic acids, such as oleic acid, palmitic acid, myristic acid, and water-insoluble oils such as benzyl benzoate, ethoxylated castor oil, palm oil, ethyl oleate, triacetin, ethyl laureate, triethyl citrate, polyethylene glycol (PEG)300, dimethylacetamide (DMA), and any combination thereof. In some embodiments, the release modifier is a hydrophobic carboxylic acid. In some embodiments, the hydrophobic carboxylic acid is oleic acid.

In some embodiments of any of the above aspects, the effective amount of the oligopeptide is 50 mg. In some embodiments, the formulation includes the oligopeptide at a concentration of 200 mg/mL and/or the oligopeptide includes 15-45% (w/w)(e.g., 15% 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45%) of a total mass of the formulation.

In some embodiments of any of the above aspects, the formulation includes the biocompatible polymer and the oligopeptide in a weight ratio of the biocompatible polymer to the oligopeptide of between 1:2 and 1:3 (e.g., 1:2 or 1:3, among others). In some embodiments, the formulation includes the biocompatible polymer and the oligopeptide in a weight ratio of the biocompatible polymer to the oligopeptide of 1:2. In some embodiments, the formulation includes the biocompatible polymer and the oligopeptide in a weight ratio of the biocompatible polymer to the oligopeptide of 1:3.

In some embodiments, the formulation is provided in an injectable volume. In some embodiments, the injectable volume is 0.5-2 mL (e.g., 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, or 1.9 mL).

In some embodiments, the formulation provides injectability of the formulation into a host through a needle ranging in diameter from 20 to 25 gauge (e.g., 20, 21, 22, 23, 24, or 25 gauge).

In some embodiments, wherein the formulation exhibits a storage cryostability including a period of at least two years (e.g., at least 25 months, at least 26 months, at least 27 months, at least 28 months, at least 29 months, at least 30 months, at least 35 months, at least 40 months, at least 45 months, at least 48 months, or more) at a temperature of 5° C. In some embodiments, the formulation exhibits stability at room temperature for up to four hours (e.g., up to 30 minutes, up to 1 hour, up to 2 hours, or up to 3 hours).

In some embodiments, the oligopeptide is homogenously dispersed within the biocompatible polymer. In some embodiments, the oligopeptide is heterogeneously dispersed within the biocompatible polymer.

In some embodiments, wherein the oligopeptide is further defined by the amino acid sequence of SEQ ID NO: 2. In some embodiments, the oligopeptide is further defined by the amino acid sequence of SEQ ID NO: 6. In some embodiments, In some embodiments, the oligopeptide is further defined by the amino acid sequence of SEQ ID NO: 7. In some embodiments, the oligopeptide is further defined by the amino acid sequence of SEQ ID NO: 9. In some embodiments, the oligopeptide is further defined by the amino acid sequence of SEQ ID NO: 10. In some embodiments, the oligopeptide is further defined by the amino acid sequence of SEQ ID NO: 11. In some embodiments, the oligopeptide is further defined by the amino acid sequence of SEQ ID NO: 12. In some embodiments, the oligopeptide is further defined by the amino acid sequence of SEQ ID NO: 13.

In some embodiments, the oligopeptide is a free base form of the oligopeptide.

In some embodiments, the oligopeptide is an acid addition salt form of the oligopeptide. In some embodiments, the acid addition salt form of the oligopeptide is selected from the group consisting of an acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts.

In some embodiments, the formulation has an intrinsic viscosity of between 0.1-0.9 dL/g (e.g., 0.2-0.8, 0.3-0.7m, 0.4-0.6, or 0.5-0.55 dL/g).

In another aspect, the disclosure provides a method of treating a subject identified as having or at risk of developing vascular dementia including administering to the subject the extended-release gel formulation of any of the foregoing aspects and embodiments.

In some embodiments, the formulation is administered to the subject at a dosage of the oligopeptide of between 10-14 mg/day (e.g., 10, 11, 12, 13, or 14 mg/day), or approximately 70 mg/week, 140 mg/two weeks, or 280 mg/month.

In some embodiments, the formulation is administered once daily, once weekly, biweekly, bimonthly, or once monthly.

In some embodiments, the formulation is administered to the subject through a needle having a diameter of 20 to 25 gauge (e.g., 20, 21, 22, 23, 24, or 25 gauge).

In some embodiments, the formulation is administered to the subject by way of subcutaneous injection or intramuscular injection.

In another aspect, the present disclosure provides a method of preparing an injectable solution including an extended-release gel formulation including at least one biocompatible polymer including PLA or PLGA having dispersed therein an effective amount of an oligopeptide of SEQ ID NO: 1, the method including providing a first sterile syringe including a first solution including at least one biocompatible polymer including PLA or PLGA and a first solvent, providing a second sterile syringe including a second solution including an effective amount of an oligopeptide of SEQ ID NO: 1 and a second solvent, admixing the first solution and the second solution by joining the first syringe and the second syringe together and injecting the first solution in the first sterile syringe into the second syringe, wherein the admixing produces the extended-release gel formulation, and decoupling the first syringe and the second syringe.

In some embodiments, the first solution includes biocompatible polymer in an amount of 50-500 mg.

In some embodiments, the first solvent and/or the second solvent is DMSO, BzOH, NMP, BB, or any combination thereof. In some embodiments, the first solution and/or the second solution further includes a release modifier. In some embodiments, the release modifier is selected from the group consisting of hydrophobic carboxylic acids, such as oleic acid, palmitic acid, myristic acid, and water-insoluble oils such as benzyl benzoate, DMA, ethoxylated castor oil, palm oil, ethyl oleate, triacetin, ethyl laureate, triethyl citrate, polyethylene glycol (PEG-)300, and any combination thereof.

In some embodiments, a combined volume of the first solution and the second solution is 1 mL.

In another aspect, the present disclosure provides a method of preparing an injectable solution including an extended-release gel formulation including at least one biocompatible polymer including PLA or PLGA having dispersed therein an effective amount of an oligopeptide of SEQ ID NO: 1, the method including providing a first sterile syringe including a powder including at least one biocompatible polymer including PLA or PLGA, providing a second sterile syringe including a first solution including an effective amount of an oligopeptide of SEQ ID NO: 1 and a solvent, admixing the powder and the solution by joining the first syringe and the second syringe together and injecting the solution in the first sterile syringe into the second syringe, wherein the admixing produces the extended-release gel formulation, and decoupling the first syringe and the second syringe.

In some embodiments, the powder includes biocompatible polymer in an amount of 50-500 mg.

In some embodiments, the solvent is DMSO, BzOH, NMP, BB, or any combination thereof. In some embodiments, the solution further includes a release modifier. In some embodiments, the release modifier is selected from the group consisting of hydrophobic carboxylic acids, such as oleic acid, palmitic acid, myristic acid, and water-insoluble oils such as benzyl benzoate, DMA, ethoxylated castor oil, palm oil, ethyl oleate, triacetin, ethyl laureate, triethyl citrate, polyethylene glycol 300, and any combination thereof.

In some embodiments, a combined volume of the solution is 1 mL.

In another aspect, the present disclosure provides a method of preparing an injectable solution including an extended-release gel formulation including at least one biocompatible polymer including PLA or PLGA having dispersed therein an effective amount of an oligopeptide of SEQ ID NO: 1, the method including providing a first sterile syringe including a solution including at least one biocompatible polymer including PLA or PLGA and a solvent, providing a second sterile syringe including a powder including an effective amount of an oligopeptide of SEQ ID NO: 1, admixing the solution and the powder by joining the first syringe and the second syringe together and injecting the solution in the second sterile syringe into the first syringe, wherein the admixing produces the extended-release gel formulation, and decoupling the first syringe and the second syringe.

In some embodiments, the solution includes the biocompatible polymer in an amount of 50-500 mg.

In some embodiments, the solvent is DMSO, BzOH, NMP, BB, or any combination thereof. In some embodiments, the solution further includes a release modifier. In some embodiments, the release modifier is selected from the group consisting of hydrophobic carboxylic acids, such as oleic acid, palmitic acid, myristic acid, and water-insoluble oils such as benzyl benzoate, DMA, ethoxylated castor oil, palm oil, ethyl oleate, triacetin, ethyl laureate, triethyl citrate, polyethylene glycol 300, and any combination thereof.

In some embodiments, a combined volume of the solution is 1 mL.

In another aspect, the present disclosure provides a method of preparing an injectable solution including an extended-release gel formulation including at least one biocompatible polymer including PLA or PLGA having dispersed therein an effective amount of an oligopeptide of SEQ ID NO: 1, the method including providing a first sterile syringe including a powder including at least one biocompatible polymer including PLA or PLGA and an effective amount of an oligopeptide of SEQ ID NO: 1, providing a second sterile syringe including a solution including and a solvent, admixing the powder and the solution by joining the first syringe and the second syringe together and injecting the solution in the first sterile syringe into the second syringe, wherein the admixing produces the extended-release gel formulation, and decoupling the first syringe and the second syringe.

In some embodiments, the powder includes the biocompatible polymer in an amount of 50-500 mg.

In some embodiments, the solvent is DMSO, BzOH, NMP, BB, or any combination thereof. In some embodiments, the solution further includes a release modifier. In some embodiments, the release modifier is selected from the group consisting of hydrophobic carboxylic acids, such as oleic acid, palmitic acid, myristic acid, and water-insoluble oils such as benzyl benzoate, DMA, ethoxylated castor oil, palm oil, ethyl oleate, triacetin, ethyl laureate, triethyl citrate, polyethylene glycol 300, and any combination thereof.

In some embodiments, a combined volume of the solution is 1 mL.

In another aspect, the present disclosure provides a method of preparing an injectable solution including an extended-release gel formulation including at least one biocompatible polymer including PLA or PLGA having dispersed therein an effective amount of an oligopeptide of SEQ ID NO: 1, the method including admixing a powder including at least one biocompatible polymer including PLA or PLGA, a powder including an effective amount of an oligopeptide of SEQ ID NO: 1, and a solution including a solvent, wherein the admixing produces the extended-release gel formulation, and adding the extended-release gel formulation into a syringe.

In some embodiments, the solution includes the biocompatible polymer in an amount of 50-500 mg.

In some embodiments, the solvent is DMSO, BzOH, NMP, BB, or any combination thereof. In some embodiments, the solution further includes a release modifier. In some embodiments, the release modifier is selected from the group consisting of hydrophobic carboxylic acids, such as oleic acid, palmitic acid, myristic acid, and water-insoluble oils such as benzyl benzoate, DMA, ethoxylated castor oil, palm oil, ethyl oleate, triacetin, ethyl laureate, triethyl citrate, polyethylene glycol 300, and any combination thereof.

In some embodiments, a combined volume of the solution is 1 mL.

In some embodiments, the formulation includes PLA in an amount of 100% of a total number of monomers in the biocompatible polymer and the biocompatible polymer has a molecular weight of between 10,000 and 18,000 Daltons (e.g., 11,000-17,000; 12,000-16,000; 13,000-15,000; or 13,500-14,500 Daltons).

In some embodiments, the formulation includes PLA in an amount of 75% and polyglycolic acid (PGA) in an amount of 25% of a total number of monomers in the biocompatible polymer and the biocompatible polymer has a molecular weight of between 4,000 and 15,000 Daltons (e.g., 5,000-14,000; 6,000-13,000; 7,000-12,000; 8,000-11,000; or 9,000-10,000 Daltons).

In some embodiments, the formulation includes PLA in an amount of 50% and PGA in an amount of 50% of a total number of monomers in the biocompatible polymer and the biocompatible polymer has a molecular weight of between 7,000 and 17,000 Daltons (e.g., 8,000-16,000; 9,000-15,000; 10,000-14,000; 11,000-13,000; or 12,000-12,500 Daltons).

In some embodiments, the effective amount of the oligopeptide is 50 mg.

In some embodiments, the biocompatible polymer and the oligopeptide have a weight ratio of the biocompatible polymer to the oligopeptide of between 1:2 and 1:3.

In some embodiments, the formulation provides injectability of the formulation into a host through a needle ranging in diameter from 20 to 25 gauge (e.g., 20, 21, 22, 23, 24, or 25 gauge).

In some embodiments, the oligopeptide is further defined by the amino acid sequence of any one of SEQ ID NOs: 2 and 6-13.

In some embodiments, the oligopeptide is a free base form of the oligopeptide.

In some embodiments, the oligopeptide is an acid addition salt form of the oligopeptide.

In some embodiments, the acid addition salt form of the oligopeptide is selected from the group consisting of acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts.

In another aspect, the present disclosure provides a kit including the extended-release gel formulation of any one of the foregoing aspects and embodiments or the syringe, or the first and second syringe of the foregoing aspects and embodiments, and a package insert.

In some embodiments, the wherein the package insert instructs a user to perform the method of any one of the foregoing aspects and embodiments

### BRIEF DESCRIPTION OF THE DRAWINGS

FIGS. 1A and 1B show photographs of exemplary extended-release gel formulations containing angiotensin-(1-7)(ANG-(1-7); SEQ ID NO: 1) oligopeptide and poly-(lactic acid)(PLA) polymer or PLA-poly-(glycolic acid (PGA))(PLGA) co-polymer in solution. FIG. 1A shows a photograph of ANG-(1-7) and PLA/PLGA in solvent containing dimethyl sulfoxide (DMSO) and benzoic acid (BzOH) at a 1:1 ratio prepared by separately dissolving ANG-(1-7) and PLGA in separate solvent solutions. Upon dissolving the ANG-(1-7) oligopeptide in solvent, the solution changed appearance from clear to hazy one day after mixing. PLGA solutions were clear before combining with ANG-(1-7). The solution containing the gel and peptide was hazy and viscous after the dissolved peptide and dissolved PLGA solutions were mixed. The gel polymers tested included 50% acid-capped PLA and 50% acid-capped PGA (RG502H), 50% ester-capped PLA and 50% ester-capped PGA (RG502S), 75% acid-capped PLA and 25% acid-capped PGA (RG752H), 75% ester-capped PLA and 25% ester-capped PGA (RG752S), and 100% acid-capped PLA (RG202H). For in vitro release (IVR) experiments, 100 μL of the RG502H, RG502S, RG752H, RG752S, and RG202H gel formulations containing ANG-(1-7) oligopeptide were added to 10 mL PBS. These formulations formed a gel depot upon addition to PBS (FIG. 1B).

FIGS. 2A-2D show elution plots produced by reverse phase high-performance liquid chromatography (RP-HPLC) analytical method for determining ANG-(1-7) oligopeptide concentration and purity from a sustained release gel formulation of the disclosure. ANG-(1-7) elution times were tested at various conditions, including 25° C. operating temperature with ultraviolet (UV) detection at 220 nm (FIG. 2A; retention time=19.26 minutes), 40° C. operating temperature with UV detection at 220 nm (FIG. 2B; retention time=18.79 minutes), and 60° C. operating temperature with UV detection at 220 nm (FIG. 2C; retention time=17.88 minutes). The area under the curve of the above-elution plots showed a linear relationship with concentration of ANG-(1-7) within the gel formulation (FIG. 2D), suggesting that RP-HPLC is an effective analytical method for measuring the concentration and purity of ANG-(1-7) oligopeptide in sustained release gel formulations disclosed herein.

FIGS. 3A-3C show plots of IVR properties of ANG-(1-7) sustained release formulations having release properties suitable for once-weekly administration. IVR is represented as percent cumulative release of ANG-(1-7) oligopeptide over time. Three hydrophobic polymer formulations of ANG-(1-7) showed release profiles that range from a couple of days to one week (RG752H and RG752S). The formulations contained 37% (w/w) PLGA. The solvent used in the formulation was 75% (v/v) DMSO with either 25% (v/v) benzyl alcohol (BA) or 25% (v/v) benzyl benzoate (BB)(FIG. 3A). IVR profile for the short-acting RG502H and RG502S polymer formulations of ANG-(1-7) showed release profiles that range from a 1-2 weeks. The formulations contained 13% of ANG-(1-7) and 37% (w/w) PLGA. The solvent used in the formulation was DMSO containing two equivalents of oleic acid (molar equivalents to ANG-(1-7))(FIG. 3B). IVR profile for the short-acting RG502H polymer formulations of ANG-(1-7) showed release profiles that range from a one to two weeks. The solvent used in the formulation was DMSO containing four equivalents of oleic acid (molar equivalents to ANG-(1-7))(FIG. 3C).

FIGS. 4A-4E show plots of IVR properties of ANG-(1-7) sustained release formulations having release properties suitable for once-monthly administration. IVR is represented as percent cumulative release of ANG-(1-7) oligopeptide over time. Three hydrophobic polymer formulations of ANG-(1-7) showed release profiles that range from two weeks to one month release (RG752H, RG752S, and RG202H)(FIG. 4A). IVR profile for the hydrophobic polymer formulation, RG752S, of ANG-(1-7) showed release profiles that range from two weeks to one month release. These formulation compositions contained two equivalents (molar ratio to ANG-(1-7)) of the excipients myristic acid or palmitic acid in the DMSO solution (FIG. 4B). IVR profile for the hydrophobic polymer formulation, RG752S, of ANG-(1-7) containing various equivalents (eq) of oleic acid (OA; molar equivalents to ANG-(1-7)) in DMSO solution. These formulations showed a two to four-week IVR profile (FIG. 4C). IVR profile for the hydrophobic polymer formulation, RG752H, of ANG-(1-7) containing two equivalents of OA (molar equivalents to ANG-(1-7)) in DMSO solution. This formulation showed a two to four-week IVR profile (FIG. 4D). IVR profile for the hydrophobic polymer formulation, RG752H, of ANG-(1-7) containing four equivalents of OA (molar equivalents to ANG-(1-7)) in the DMSO solution. This formulation showed a two to four-week IVR profile (FIG. 4E).

FIG. 5 shows a plot of IVR properties of ANG-(1-7) variant, PNA5 (SEQ ID NO: 10), sustained release formulations having release properties suitable for once-weekly administration. IVR is represented as percent cumulative release of PNA5 oligopeptide over time. IVR profile for the polymer formulation, RG502S, of PNA5 in DMSO solution showed a one-week IVR profile. IVR profile for the polymer formulation RG752S in DMSO solution containing 45 mg OA showed a one to two-week release profile.

FIG. 6 shows a plot of IVR properties of PNA5 sustained release formulations having release properties suitable for once-monthly administration. IVR is represented as percent cumulative release of PNA5 oligopeptide over time. IVR profile for the polymer formulation RG752S in DMSO solution containing 45 mg OA showed a two-week release profile. IVR profile for the polymer formulation RG752S in DMSO solution containing 25% BB showed a very long release profile of at least four weeks.

FIG. 7 shows a bar graph showing the effects of treatment with 50 mg/mL of PNA5 with RG752S in 3:2 DMSO to BB solvent in mice using a Novel Object Recognition Test (NOR). Students-t test, *=p<.05.

### DEFINITIONS

As used herein, the term “ANG-1-7 variant” refers to oligopeptide in which one or more amino acid residue is either modified or different than the amino acid residue of the corresponding native ANG-(1-7). The term “ANG-1-7 variant” also includes oligopeptide of eight amino acid residues as discussed in more detail below. Non-limiting examples of ANG-(1-7) variants are described in SEQ ID NOs: 6-13.

As used herein, the term “average rate of release” refers to the numerical average of the rate of drug release from a formulation of the disclosure over a defined period of time. According to the present disclosure, the average rate of release may be quantified as:

$$\frac{{\sum }_{0}^{n} \frac{\% of total drug released}{time interval}}{n}$$

where n=total number of time intervals.

As used herein, the term “burst release” refers to a physical property of controlled-release drug formulations characterized by an large initial release of the therapeutically active drug cargo following placement of the formulation in a release medium. Burst release results in a higher initial drug dose and may also reduce the effective lifetime of a formulation by prematurely depleting levels of the therapeutic drug upon administration. Generally, burst release may be measured from the initial time (t₀) of placement of the formulation in a release medium or administration to a subject to t₀+Δt, where Δt is a time interval ranging from 1 to 24 hours.

As used herein, the term “carbohydrate” refers to pentose and hexose of empirical formula (CH₂O)n, where n is 5 for pentose and 6 for hexose. A carbohydrate can be monosaccharide, disaccharide, oligosaccharide (e.g., 3-20, typically 3-10, and often 3-5 monomeric saccharides are linked together), or polysaccharide (e.g., greater than 20 monomeric saccharide units). The term carbohydrate may refer specifically to a monosaccharide and/or disaccharide. However, it should be appreciated that the scope of the invention is not limited to mono- or di-saccharides. The terms “carbohydrate” and “saccharide” are used interchangeably herein.

As used herein, the term “cognitive impairment” refers to a broad set of neurological conditions and disorders characterized by any impairment to cognition beyond those expected based on an individual's age and education. Cognitive impairment may refer to impairment to any one of the following cognitive functions, including, but not limited to, memory, learning, comprehension and/or production of language, visuospatial coordination, executive function, judgement, evaluation, reasoning, problem solving, decision making, metacognition, and impulse control. Various neurological disorders may feature cognitive impairment as a central or peripheral symptom, such as, e.g., dementia (e.g., vascular dementia, mild cognitive impairment, age-related cognitive decline, Alzheimer's disease, frontotemporal dementia, progressive supranuclear palsy, corticobasal degeneration, prior disease, alcohol-related dementia, mixed dementia, or a dementia that is a secondary co-morbidity to another disease or condition (e.g., dentatorubral-pallidoluysian atrophy, epilepsy, fatal insomnia, Fragile-X-associated tremor/ataxia syndrome, glutaric aciduria type 1, leukodystrophy, maple syrup urine disease, Niemann-Pick disease type C, neuronal ceroid lipofuscinosis, neuroacanthocytosis, organic acidemia, Sanfilippo syndrome type B, spinocerebellar ataxia type 2, or urea cycle disorder)).

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gin | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “derivative” refers to any chemical modification of the amino acid, such as alkylation (e.g., methylation or ethylation) of the amino group or the functional group on the side chain, removal of the side-chain functional group, addition of a functional group (e.g., hydroxyl group on proline), attachment of mono- or di-carbohydrate (e.g., via glycosylation) etc. Exemplary glycosylated derivatives include hydroxyl group on serine that is glycosylated with glucose, galactose, ribose, arabinose, xylose, lyxose, allose, altrose, mannose, gulose, iodose, talose, fucose, rhamnose, etc., as well as disaccharides and amino sugars such as galactosamine, glucosamine, sialic acid, N-acetyl glucosamine, etc. Amino acid derivatives also include modified or unmodified D-amino acids.

As used herein, the terms “effective amount,” “therapeutically effective amount,” and “sufficient amount” of a formulation or composition described herein refer to a quantity sufficient to, when administered to the subject effect beneficial or desired results, including clinical results, and, as such, an “effective amount” or synonym thereto depends upon the context in which it is being applied. For example, in the context of treating a cognitive impairment (e.g., vascular dementia), it is an amount of the formulation or composition sufficient to achieve a treatment response as compared to the response obtained without administration of the formulation or composition. The amount of a given formulation or composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art by routine methods known in the art. Dosage regimen may be adjusted to provide the optimum therapeutic response.

As used herein, the term “extended-release” refers to a physical property of a pharmaceutical formulation of the disclosure such that the formulation is configured to gradually release a steady amount of a biologically-active therapeutic agent dispersed therein over a defined period of time (e.g., over a period of a day, week, month, etc.). Extended-release formulation offer benefits for the delivery of therapeutic agents to patients by allowing the formulation to provide continuous dosing of the therapeutic agent in the subject without requiring frequent administration. Thus, extended-release formulations mitigate the problems associated with delivering a high dose of a drug to a patient and improve patient compliance. According to the present disclosure, extended-release can be defined as a percent (%) release of a total dose of a therapeutic agent (e.g., oligopeptides of the disclosure) contained in an extended-release formulation over a defined time interval. Furthermore, extended-release may also require that the rate of release of a drug from a formulation of the disclosure does not exceed a specific rate of release within a defined period of time (e.g., rate of drug release does not exceed, e.g., X %/Y hours).

As used herein, the terms “high performance liquid chromatography” and “HPLC” refer to an analytical method for the separation of one or more analytes in a mixture by passing the mixture in a solution or in a suspension (i.e., mobile phase) through a medium (i.e., stationary phase) in which the analytes of the mixture move at different rates. HPLC may employ a polar stationary phase (e.g., silica beads) for the purification of charged analytes or a non-polar stationary phase for the separation of hydrophobic analytes. The general principle of HPLC is that analytes that more strongly interact with the adsorbent stationary phase will elute later, while those that interact less strongly with the stationary phase will elute earlier, thereby enabling separation and identification of analytes in a mixture.

As used herein, the term “inverso modified” refers to a peptide which is made up of D-amino acids in which the amino acid residues are assembled in the same direction as the native peptide with respect to which it is inverso modified.

As used herein, “locally” or “local administration” means administration at a particular site of the body intended for a local effect and not a systemic effect. Examples of local administration are epicutaneous, inhalational, intra-articular, intrathecal (e.g., lumbar puncture), intravaginal, intravitreal, intrauterine, intralesional administration, lymph node administration, intratumoral administration, and administration to the central nervous system (CNS)(e.g., intraparenchymal, intracerebroventricular, or intrathecal), wherein the administration is intended to have a local and not a systemic effect.

As used herein, the terms “mass spectrometry” and “MS” refer to an analytical technique used to determine that mass-to-charge ratio of ions in a sample. MS has a number of applications, including identification of unknown compounds, determination of isotopes in a molecule, determination of compound structure. MS is frequently used for protein characterization and sequencing by way of electrospray ionization (ESI) or matrix-assisted laser desorption/ionization (MALDI).

As used herein, the term “native” refers to any sequence of L amino acids used as a starting sequence or a reference for the preparation of partial or complete retro, inverso or retro-inverso analogues. Thus, the term “native ANG-(1-7)” refers to an oligopeptide having the same amino acid sequence as that of endogenous ANG-(1-7). It should be appreciated that the use of the term “native” does not imply naturally-occurring, although it can include the endogenous form of ANG-(1-7). The term “native” merely refers to having the same amino acid sequence as that of endogenous Ang-(1-7) without any modification of the amino acid residues. Accordingly, the term “native ANG-(1-7)” includes both synthetic ANG-(1-7) and endogenous ANG-(1-7) as long as the amino acid residues are the same and are not modified.

As used herein, the term “oligopeptide” refers to an amino acid chain of any length, but typically amino acid chain of about fifteen or less amino acids, ten or less amino acids, eight or less amino acids, or seven or eight amino acids. An exemplary oligopeptide includes the ANG-(1-7) heptapeptide or a variant thereof (e.g., any one of SEQ ID NOs: 6-13). It should be appreciated that one or more of the amino acids of ANG-(1-7) can be replaced with an “equivalent amino acid”, e.g., L (leucine) can be replaced with isoleucine or other hydrophobic side-chain amino acid such as alanine, valine, methionine, etc., and amino acids with polar uncharged side chain can be replaced with other polar uncharged side chain amino acids. While ANG-(1-7) comprises 7 amino acids, in some embodiments the oligopeptide of the disclosure has eight or less amino acids.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, e.g., using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject without excessive toxicity, irritation, allergic response and other problem complications commensurate with a reasonable benefit/risk ratio.

As used herein, the terms “poly-glycolic acid,” “polyglycolide,” and “PGA” refer to a polymeric form of glycolic acid of variable lengths and weights. PGA is a biodegradable, thermoplastic polymer that is commonly used in medicinal suture and drug delivery systems. PGA can be incorporated into a polymer matrix containing poly-lactic acid (PLA) to produce poly-(lactic co-glycolic acid)(PLGA).

As used herein, the terms “poly-lactic acid,” “polylactide,” and “PLA” refer to a thermoplastic polyester containing a variable number of lactic acid monomers. PLA has been employed in a number of commercially available products, including medical implants, drug delivery systems, and decomposable materials. PLA can be incorporated into a polymer matrix containing PGA, i.e., PLGA.

As used herein, the terms “poly-(lactic co-glycolic acid)” or “PLGA” refer to a copolymer containing PGA and PLA at various ratios. PLGA can be synthesized as a random or block copolymer to impart specific properties. Generally, higher PGA content in the PLGA copolymer will result in improved hydrolysis of PLGA. PLGA or monomeric components thereof may be end-capped with esters (ester-capped) or with a free carboxylic acid (acid-capped). PLGA is commonly used as a drug delivery vehicle owing to its biodegradability and tolerability. For example, an FDA approved Lupron depot uses PLGA as a drug vehicle for the treatment of advanced prostate cancer.

As used herein, the term “retro modified” refers to a peptide which is made up of L-amino acids in which the amino acid residues are assembled in opposite direction to the native peptide with respect the which it is retro modified.

The term “retro-inverso modified” refers to a peptide which is made up of D-amino acids in which the amino acid residues are assembled in the opposite direction to the native peptide with respect to which it is retro-inverso modified. Thus, native Ang-(1-7) (L-amino acids, N→C direction) is: Asp-Arg-Val-Tyr-Ile-His-Pro, i.e., DRVYIHP (SEQ ID NO: 2). Retro-inverso ANG-(1-7) (D-amino acids, C→N direction) is: DRVYIHP (SEQ ID NO: 3). Retro ANG-(1-7) (L-amino acids, C→N direction) is: DRVYIHP (SEQ ID NO: 4). Finally, inverso ANG-(1-7) (D-amino acids, N→C direction) is: DRVYIHP (SEQ ID NO: 5). The use of D-amino acids in the context of inverso modified and retro-inverso modified ANG-(1-7) derivatives is not intended to be limiting on the use of D-amino amino acids in the oligopeptides. As discussed in more detail below, fewer than all of the amino acids in an ANG-(1-7) derivative may be D-amino acids.

As used herein, the terms “subject” and “patient” refer to a mammal (e.g., human). A subject to be treated according to the methods described herein may be one who has been diagnosed with a cognitive impairment (e.g., vascular dementia). Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “sucrose acetoisobutyrate” and “SAIB” refer to an additive used for various purposes, including additives in beverages, color cosmetics and skin care, flavorings, fragrances, hair products, and horse styling products. Within the context of the present disclosure, SAIB is used as a biocompatible polymer suitable for producing extended-release gel formulations for sustained release of one or more of the therapeutic peptides disclosed herein (e.g., ANG-(1-7) or PNA5). SAIB has the chemical structure shown below.

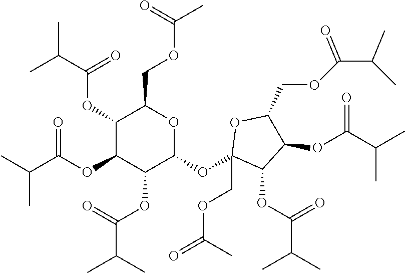

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “variant,” when referring to an oligopeptide of the present disclosure, refers to an oligopeptide having one or more modifications made to any one of its amino acid residues (e.g., methylation, addition of a functional group (e.g., hydroxy group on proline), glycosylation, replacement with a D-amino acid or an equivalent amino acid, and/or the terminal amino group end or the carboxyl end of the oligopeptide is modified (e.g., carboxylic end can be modified to an amide, amine, thiol, or alcohol, or an additional amino acid residue is added).

As used herein, the term “vascular dementia” refers to a cerebrovascular dementia caused by insufficient supply of blood to the brain, typically resulting from a series of minor ischemic or hemorrhagic strokes, that eventually leads to lesions of central nervous system (CNS) tissue and cognitive decline. Patients suffering from vascular dementia often experience cognitive decline, motor deficits, abnormal behavior, dysregulated affect, hemiparesis, bradykinesia, hyperreflexia, extensor plantar reflexes, ataxia, pseudobulbar palsy, gait problems, and swallowing difficulties. Generally, vascular dementia can result from one or more minor strokes in different parts of the CNS, including anterior cerebral artery territory, parietal lobes, cingulate gyrus, hippocampus, and/or thalamus.

### DETAILED DESCRIPTION

Disclosed herein are formulations comprising an extended-release gel matrix containing an angiotensin-(1-7) oligopeptide (ANG-(1-7)) or a variant thereof and a biocompatible polymer. In particular, the disclosure features extended-release gel formulations containing a poly-lactic acid (PLA), poly-(lactic-co-glycolic acid)(PLGA) polymer matrix, or sucrose acetoisobutyrate (SAIB) polymer having dispersed therein an effective amount (e.g., a therapeutically effective amount) of ANG-(1-7) oligopeptide or a variant thereof. The formulation also contains a solvent (e.g., an organic solvent) for dissolving the biocompatible polymer and the ANG-(1-7) oligopeptide or a variant thereof. The formulation may further be suspended in an injection vehicle suitable for administration to a subject (e.g., a human) using syringe diameters (e.g., 20-25 gauge) appropriate for administration to the subject (e.g., by way of intramuscular or subcutaneous injection). Also disclosed are methods of treating or preventing vascular dementia in a subject identified as having or at risk of developing vascular dementia by administering to the subject a formulation of the disclosure. An important benefit to the disclosed formulations is their ability to exhibit extended-release over a time period ranging from one week to one month, their mitigation of an initial burst release that often leads to undesirable spikes in drug dose at the start of administration, and their superior storage and in-use stability.

### Angiotensin-(1-7) Oligopeptide and Variants Thereof

The renin angiotensin system (RAS) involves two separate enzymatic pathways providing a physiological counterbalance of two related peptides acting at distinct receptors. The ACE-AngII-AT1 receptor system is thought to be physiologically opposed and balanced by the ACE2-ANG-(1-7)-Mas system. Functionally, these two separate enzymatic pathways of RAS are thought to be involved in balancing reactive oxygen species (ROS) production and nitric oxide (NO) in the brain, microvasculature and peripheral tissues. Increases in AT1 receptor activation are known to increase NAD(P)H oxidase and ROS generation which are both known to contribute to abnormal increases of sympathetic nerve activity observed in CHF and hypertension. This increase in AT1 receptor-induced ROS formation is thought to be opposed by ACE2-ANG-(1-7)-Mas inhibition of ROS formation. ANG-1-7, the majority of which is produced from ACE2 cleavage of Ang II, decreases ROS production and increases NOS in the brain via activation of Mas and, possibly, through the AT2 receptor.

Within the brain, the Mas receptor is known to be expressed on neurons, microglia and vascular endothelial cells. Further, the key components that make up the “neurovascular unit” (i.e., neurons, microglia, and endothelial cells) are important players in brain inflammation and ROS production. The end-result of this feed-forward cascade is neuronal dysfunction and cognitive impairment. The ideal therapeutic candidate to treat cognitive impairment would be designed to interrupt this cascade by working at both sides of the blood-brain barrier, the brain vascular endothelium and neuronal cells. ANG-1-7, acting at the Mas receptor, is known to have effects at both endothelial cells and neurons.

According to the present disclosure, the ANG-(1-7) oligopeptide may be an oligopeptide having the general formula:

(SEQ ID NO: 1)
A¹-A²-A³-A⁴-A⁵-A⁶-A⁷-A⁸;

where A¹ is selected from the group consisting of aspartic acid, glutamic acid, alanine, and a derivative thereof; A² is selected from the group consisting of arginine, histidine, lysine, and a derivative thereof; A³ is selected from the group consisting of valine, alanine, isoleucine, leucine, and a derivative thereof; A⁴ is selected from the group consisting of tyrosine, phenylalanine, tryptophan, and a derivative thereof; A⁵ is selected from the group consisting of isoleucine, valine, alanine, leucine, and a derivative thereof; A⁶ is selected from the group consisting of histidine, arginine, lysine, and a derivative thereof; A⁷ is selected from the group consisting of proline, glycine, serine, and a derivative thereof; and A⁸ can be present or absent, wherein when A⁸ is present, A⁸ is selected from the group consisting of serine, threonine, hydroxyproline, and a derivative thereof, provided (i) at least one of A¹-A⁸ is optionally substituted with a mono- or di-carbohydrate; or (ii) when A⁸ is absent: (a) at least one of A⁷-A⁷ is substituted with a mono- or di-carbohydrate, (b) A⁷ is terminated with an amino group, or (c) a combination thereof. In some embodiments, the carbohydrate includes glucose, galactose, xylose, fucose, rhamnose, lactose, cellobiose, melibiose, or a combination thereof. In some embodiments, A⁸ is serine or a variant thereof.

In some embodiments, (i) A⁸ is terminated with an amino group; or (ii) when A⁸ is absent, A⁷ is terminated with an amino group. In some instances, (i) A⁸ is serine that is glycosylated with glucose or lactose; or (ii) when A⁸ is absent, A⁷ is serine that is glycosylated with glucose or lactose. In other instances, when A⁸ is absent and A⁷ is serine that is glycosylated with glucose. In some instances, in some cases A⁷ is terminated with an amino group.

In other embodiments, A¹ is aspartic acid; A² is arginine; A³ is valine; A⁴ is tyrosine; A⁵ is isoleucine; A⁶ is histidine; and (i) A⁸ is absent and A⁷ is terminated with an amino group or A⁷ is a glycosylated serine, or (ii) A⁸ is serine terminated with an amino group. In some embodiments, A⁸ is a glycosylated serine. In other embodiments, A⁸ is absent and A⁷ is a glycosylated serine that is terminated with an amino group.

Further still, the ANG-(1-7) oligopeptide may be a variant having eight amino acids or less (e.g., 7 or 8 amino acid residues). In some embodiments, the ANG-(1-7) oligopeptide is glycosylated, e.g., with xylose, fucose, rhamnose, glucose, lactose, cellobiose, melibiose, or a combination thereof. In some embodiments, the carboxylic acid end of a glycosylated ANG-(1-7) oligopeptide is substituted with an amino group.

Accordingly, the ANG-(1-7) oligopeptide may be a native ANG-(1-7) heptapeptide having an amino acid sequence of (L-amino acids, N→C direction) is: Asp-Arg-Val-Tyr-Ile-His-Pro, i.e., DRVYIHP (SEQ ID NO: 2). In other embodiments, the ANG-(1-7) oligopeptide may be a retro-inverso ANG-(1-7) oligopeptide having an amino acid sequence of (D-amino acids, C→N direction) is: DRVYIHP (SEQ ID NO: 3). In other embodiments, the ANG-(1-7) oligopeptide may be a retro ANG-(1-7) oligopeptide having an amino acid sequence of (L-amino acids, C→N direction) is: DRVYIHP (SEQ ID NO: 4). In still further embodiments, the ANG-(1-7) oligopeptide may be an inverso ANG-(1-7) oligopeptide having an amino acid sequence of (D-amino acids, N→C direction) is: DRVYIHP (SEQ ID NO: 5).

In some examples of the present disclosure, the ANG-(1-7) oligopeptide of the disclosure may be any one of the oligopeptides described in Table 2, as is shown below.

TABLE 2
Exemplary angiotensin-(1-7) oligopeptides and variants thereof

| ANG-(1-7) | Amino acid residue | | | | | | | | Carboxyl terminal |
|---|---|---|---|---|---|---|---|---|---|
| Oligopeptide | 1 | 2 | 3 | 4 | 5 | 6 | 7 | 8 | end functional group |
| Native ANG-(1-7) | Asp | Arg | Val | Tyr | Ile | His | Pro | — | OH (SEQ ID NO: 2) |
| ANG-(1-7)_V1 | Asp | Arg | Val | Tyr | Ile | His | Pro | — | NH₂ (SEQ ID NO: 6) |
| ANG-(1-7)_V2 | Asp | Arg | Val | Tyr | Ile | His | Pro | Ser° | NH₂ (SEQ ID NO: 7) |
| ANG-(1-7)_V3 | Asp | Arg | Val | Tyr | Ile | His | Pro | Ser* | NH₂ (SEQ ID NO: 8) |
| ANG-(1-7)_V4 | Asp | Arg | Val | Tyr | Ile | His | Pro | Ser** | NH₂ (SEQ ID NO: 9) |
| ANG-(1-7)_V5 | Asp | Arg | Val | Tyr | Ile | His | Ser* | — | NH₂ (SEQ ID NO: 10) |
| (PNA5) | | | | | | | | | |
| ANG-(1-7)_V6 | Ala | Xxx | Yyy | Tyr | Ile | Zzz | Pro | Ser°*/** | NH₂ (SEQ ID NO: 11) |
| ANG-(1-7)_V7 | Asp | Arg | Xxx | Tyr | Yyy | His | Pro | Ser°*/** | NH₂ (SEQ ID NO: 12) |
| ANG-(1-7)_V8 | Asp | Arg | Xxx | Zzz | Yyy | His | Pro | Ser°*/** | NH₂ (SEQ ID NO: 13) |

In Table 2, Vn (e.g., V1, V2, V3, etc.) represents an ANG-(1-7) oligopeptide variant identifier (e.g., V1 is ANG-(1-7) variant oligopeptide 1), Ser° refers to an unglycosylated serine residue, Ser* refers to a glycosylated serine residue, and Ser** refers to a lactosylated serine residue, Xxx, Yyy, and Zzz refer to any amino acid residue.

As shown in Table 2, some of the oligopeptides have carbohydrate attached to the native ANG-(1-7) peptide. These peptides are sometimes referred to as glycopeptides. Studies have shown that inherent binding of the glycopeptide to the native receptor is minimally affected. Therefore, the glycosylated ANG-(1-7) variants, at a minimum, maintain Mas binding similar to that of the native ANG-(1-7) peptide. In addition, promoting the aqueous nature of the glycopeptide can further enhance vascular efficacy of ANG-(1-7) variants. The degree of glycosylation (e.g., Table 2: unglycosylated Ser°, glucosylated Ser* or lactosylated Ser**) for optimal blood-brain barrier transport is determined using the best binding compounds from these using the in vivo mouse model. Besides the disaccharide β-lactose, the more robust disaccharide β-cellobiose is examined using these first few structures. Based on the amino acid sequence of ANG-(1-7) and the potential modification strategies, there are at least about 200 possible variants of ANG-(1-7) that are rapidly generated using the well-known oligopeptide synthesis, including automated peptide synthesis as well as combinatorial synthesis.

### Extended-Release Gel Formulations

The present disclosure features an extended-release gel formulation containing a biocompatible (co-)polymer and an effective amount of an ANG-(1-7) peptide or a variant thereof (e.g., any one of SEQ ID NOs: 1-13).

### Biocompatible Polymer

According to the compositions and methods disclosed herein, an extended-release gel formulation of the disclosure contains a biocompatible polymer (e.g., homopolymer or co-polymer). In some embodiments, polymers suitable for use in conjunction with the disclosed compositions include linear polyesters. The linear polyesters may be prepared from α-hydroxy carboxylic acids, e.g. lactic acid and/or glycolic acid, by condensation of the lactone dimers, see e.g. U.S. Pat. No. 3,773,919, the contents of which are incorporated herein by reference. The polyester chains in the linear polymers may be copolymers of the α-carboxylic acid moieties, lactic acid and glycolic acid, or of the lactone dimers.

In some embodiments, the biocompatible polymer may be sucrose acetoisobutyrate (SAIB). SAIB is an FDA-approved food additive that exhibits safe human daily intake of up to 20 mg/kg. Once in the body, SAIB is metabolized into sucrose and partially acylated sucrose, which are readily cleared from the body. SAIB is a highly hydrophobic, viscous liquid that forms a low-viscosity solution when mixed in certain organic solvents.

### Molecular Weight

Linear polyesters, e.g. linear PLGA, that may be used according to the present disclosure have a weight average molecular weight (MW) between about 10,000 and about 500,000 Da, e.g. between about 47,000 to about 63,000, between about 24,000 and 38,000, between about 10,000 and 8,000, between about 4,000 and 15,000, or between about 7,000 and 17,000. Such polymers have a polydispersity M_{w}/Mₙ e.g. between 1.2 and 2. Suitable examples include e.g. poly(D,L-lactide-co-glycolide), e.g. having a general formula —[(C₆H₈O₄)ₓ(C₄H₄O₄)_{y}]ₙ— (each of x, y and n having a value so that the total sum gives the above indicated MWs), e.g. those commercially available, e.g. Resomers® from Boehringer Ingelheim, in particular Resomers® RG, e.g. Resomer® RG 502, 502H, 503, 503H, 504, 504H.

### End-Capping

According to the present disclosure, the biocompatible polymers suitable for use with the formulations disclosed herein (e.g., PLA or PLGA) may be end-capped with esters or with a free carboxylic acid. Generally, ester-capped polymers exhibit a longer degradation half-live as compared to acid-capped polymers. In some embodiments, the biocompatible polymer of the disclosure is ester-capped. In some embodiments, the biocompatible polymer of the disclosure is acid-capped (e.g., carboxylic acid-capped).

### Co-Polymer Ratio

The molar ratio of lactide:glycolide of PLGA in the formulation of the disclosure may be from about 100:0 to 25:75 (e.g., 100:0, 90:10, 80:20, 75:25, 60:40, 50:50, 40:60, 30:70, or 25:75). In some embodiments, the molar ratio of lactide:glycolide in a PLGA co-polymer is 100:0 (e.g., PLA), 75:25, or 50:50. In some embodiments, the molar ratio of lactide:glycolide in a PLGA co-polymer is 100:0. In some embodiments, the molar ratio of lactide:glycolide in a PLGA co-polymer is 75:25. In some embodiments, the molar ratio of lactide:glycolide in a PLGA co-polymer is 50:50.

### Oligopeptide to Polymer Ratio

The present disclosure provides extended-release gel formulations containing a therapeutic ANG-(1-7) oligopeptide or a variant thereof and a biocompatible polymer (e.g., PLA or PLGA) at a given ratio. In some embodiments, the gel formulation contains an ANG-(1-7) oligopeptide or a variant thereof and a biocompatible polymer at a ratio of 3:1. In some embodiments, the gel formulation contains an ANG-(1-7) oligopeptide or a variant thereof and a biocompatible polymer at a ratio of 3:2. In some embodiments, the gel formulation contains an ANG-(1-7) oligopeptide or a variant thereof and a biocompatible polymer at a ratio of 1:1. In some embodiments, the gel formulation contains an ANG-(1-7) oligopeptide or a variant thereof and a biocompatible polymer at a ratio of 2:3. In some embodiments, the gel formulation contains an ANG-(1-7) oligopeptide or a variant thereof and a biocompatible polymer at a ratio of 1:3. In some embodiments, the gel formulation contains an ANG-(1-7) oligopeptide or a variant thereof and a biocompatible polymer at a ratio of 1:2. Other ratios of the ANG-(1-7) oligopeptide or a variant thereof and the biocompatible polymer not described herein may be used.

### Solvents

The sustained release gel formulations of the disclosure may be prepared by dissolving the biocompatible polymer (e.g., PLA or PLGA) and the biologically-active therapeutic agent (i.e., ANG-(1-7) oligopeptide) in a solvent, such as, e.g., an organic solvent or aqueous solvent. The biocompatible polymer the biologically-active therapeutic agent may be separately dissolved using the same type of solvent and then combined together. Alternatively, the biocompatible polymer and the biologically-active therapeutic agent may be dissolved together in the same admixture containing the solvent.

As the organic solvent, dimethyl sulfoxide (DMSO), oleic acid, ethoxylated castor oil, ethyl oleate, triacetin, ethyl laureate, triethyl citrate, polyethylene glycol 300, N-methyl-2-pyrrolidone (NMP), benzoic acid (BzOH), benzyl alcohol (BA), benzyl benzoate (BB), dimethylacetamide (DMA), dichloromethane, chloroform, dichloroethane, trichloroethane, carbon tetrachloride, ethyl ether, isopropyl ether, ethyl acetate, butyl acetate, benzene, toluene, xylene, ethanol, methanol, acetonitrile and the like may be used. These may be used in admixture of appropriate ratios. For example, different ratios of the above solvents may be combined to achieve optimal solubility of the biocompatible polymer and the biologically-active therapeutic agent. In a non-limiting example, DMSO and BzOH may be combined in a 1:1 (v/v) ratio to dissolve the polymer and the therapeutic agent. In another example, DMSO and BB are combined at a 75%:25% (v/v) ratio. Furthermore, certain solvents made be added as excipients. In particular, oleic acid, palmitic acid, and myristic acid may be added as excipients. Water-insoluble oils, such as, e.g., benzyl benzoate, ethoxylated castor oil, palm oil, ethyl oleate, triacetin, ethyl laureate, triethyl citrate, polyethylene glycol (PEG) 300, dimethylacetamide (DMA) may be added as excipients to primary solvents (e.g., DMSO, NMP, BB, etc.).

Relatedly, various aqueous buffers may also be used as a vehicle for testing the in vitro release properties of the disclosed formulations. Once the formulation is added to these aqueous buffers, it generally forms a gel depot from which the active ingredient (e.g., an oligopeptide disclosed herein) is released. Non-limiting examples of suitable aqueous vehicles include PBS, sterile saline solution (e.g., 0.9% NaCl solution), sterile water, lactated Ringer solution, Ringer's acetate, sodium-bicarbonate solution, among others.

### Release Kinetics

Disclosed herein are extended-release gel formulations that allow for controlled-release of ANG-(1-7) oligopeptides over timescales suitable for administration to a human subject, such that, when injected (e.g., by way of intramuscular or subcutaneous injection) produce a therapeutically-effective amount of the oligopeptide in one or more fluids (e.g., blood, serum, or plasma), cells (e.g., neurons, glial cells, endothelial cells, fibroblasts, among others), tissues (e.g., nervous tissue or vascular tissue), or organs (e.g., brain) in the subject.

### Sustained Release and Burst Release

Central to the discovery made by the inventors is that the gel formulations disclosed herein exhibit therapeutically desirable extended-release profiles that provide steady, therapeutically-effective dosing of the ANG-(1-7) oligopeptide over multiple timescales. Such extended-release formulations offer the benefit of reducing the amount of drug necessary to produce the same therapeutic effect in patients. This also reduces the requirement for frequent administration, thereby reducing the number of doses needed to be administered and improving patient compliance with the administration regimen. In particular, the inventors have discovered two types of formulations that, when added into a release medium (e.g., PBS at 37° C., pH 7.4), exhibit sustained release profiles that span from, e.g., 1 to 10 days and, e.g., 24 to 30 days, respectively.

In a particular example, the present disclosure provides an extended-release gel formulation containing a biocompatible polymer (e.g., PLA or PLGA) having dispersed therein an effective amount of an oligopeptide of SEQ ID NO: 1, such that the formulation exhibits a sustained release in vitro of at least 60% of the effective amount of the therapeutic agent within 48 hours following placement of the formulation in a release medium (e.g., PBS at 37° C., pH 7.4) at a time=0 (t₀) and an initial burst release not greater than 30% of the effective amount of the oligopeptide within 24 hours following t₀. The formulation may be configured to have a sustained release profile that does not exceed an average rate of release of the oligopeptide that is greater than 20%/24 hours for a period that is equal to or greater than seven days following t₀, as measured by high performance liquid chromatography (HPLC) and/or mass spectrometry (MS) at an operating temperature of 37° C.

In another example, the present disclosure provides an extended-release gel formulation containing a biocompatible polymer and an effective amount of an oligopeptide of SEQ ID NO: 1, such that the formulation exhibits a sustained release in vitro of at least 60% of the effective amount of the therapeutic agent within 25 days following placement of the formulation in a release medium (e.g., PBS at 37° C., pH 7.4) at a time=0 (t₀) and an initial burst release not greater than 30% of the effective amount of the oligopeptide within 24 hours following t₀. The formulation may be configured to have a sustained release profile that does not exceed an average rate of release of the oligopeptide that is greater than 30%/24 hours for a period that is equal to or greater than seven days following t₀, as measured by HPLC and/or MS at an operating temperature of 37° C.

In the context of in vivo administration to a subject (e.g., a human), the present disclosure provides an extended-release gel formulation containing a biocompatible polymer and an effective amount of a biologically-active therapeutic agent, such that, following subcutaneous or intramuscular administration to a human subject, the formulation is configured to form a depot in vivo that releases the oligopeptide at a rate sufficient to maintain an average serum concentration that is between 1-1000 ng/mL for a period of 24-168 hours following administration, a maximum serum concentration (Cₘₐₓ) of the oligopeptide that is between 1-1000 ng/mL for a period of 48 hours following administration.

In another example, the present disclosure provides an extended-release gel formulation containing a biocompatible polymer having dispersed therein an effective amount of an oligopeptide of SEQ ID NO: 1, such that, following subcutaneous or intramuscular administration to a human subject, the formulation is configured to form a depot in vivo that releases the oligopeptide at a rate sufficient to maintain an average serum concentration of between 1 and 1000 ng/mL for a period of 21 days following administration, a Cₘₐₓ of the oligopeptide of between 1 and 1000 ng/mL for a period of 21 days following administration.

Routine and well-known methods may be employed to determine in vivo pharmacokinetic parameters in human subjects.

### Syringability

Pharmaceutical forms of the disclosed extended-release gel formulations must exhibit a viscosity that is appropriate for syringability to a human subject such that a syringe needle of a reasonable diameter (e.g., a needle diameter that does not cause undue pain to the subject) may be used and dosing is uniform across multiple rounds of administration. Accordingly, the present disclosure provides gel formulations having a viscosity that is compatible with injection to a human subject using a needle diameter of 20 to 25 gauge (e.g., 20, 21, 22, 23, 24, or 25 gauge).

### Stability

The extended-release gel formulations disclosed herein offer the benefit of long-term stability of a pharmaceutical composition containing the same, therefore, eliminating a requirement for admixing the gel-formulation and a pharmaceutically-acceptable injection vehicle prior to injection to a subject. Accordingly, the present disclosure provides extended-release gel formulations containing a biocompatible polymer and an effective amount of an ANG-(1-7) oligopeptide having a cryostability of at least 24 (e.g., at least 24, 26, 28, 30, 32, 34, 36, 38, 40, 42, 44, 46, 48, or more) months at 5° C.

The disclosed gel formulations also exhibit excellent in-use stability, such that the formulation may remain stable (i.e., prevent denaturation of the oligopeptide or degradation of the biocompatible polymer) for a period of up to 4 (e.g., up to 4, 3, 2, or 1) hours at room temperature.

### Pharmaceutical Compositions

The extended-release gel formulation of the disclosure may be formulated into pharmaceutical compositions for administration to a mammalian (e.g., a human) subject in a biologically compatible form suitable for administration in vivo. The compositions disclosed herein may be formulated in any suitable vehicle for delivery to a subject. For instance, they may be formulated in a pharmaceutically acceptable suspension, dispersion, solution, or emulsion. Suitable mediums include saline preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles suitable for intravenous administration include fluid and nutrient replenishers, electrolyte replenishers (such as those based on Ringer's dextrose), and the like.

Preservatives and other additives may also be present such as, e.g., antimicrobials, antioxidants, chelating agents, and inert gases and the like. A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

Accordingly, the compositions described herein may be formulated for administration, e.g., by subcutaneous, intramuscular, oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration may be by continuous infusion over a selected period of time.

Solutions of an agent described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, e.g., in Remington's Pharmaceutical Sciences (2012, 22^{nd} Ed.) and in The United States Pharmacopeia: The National Formulary (USP 41 NF 36), 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Local, regional, or systemic administration also may be appropriate. A composition described herein may advantageously be contacted by administering an injection or multiple injections to the target site, spaced e.g., at approximately, 1 cm intervals.

The compositions described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

Accordingly, the present disclosure relates to a pharmaceutical composition containing an extended-release gel formulation disclosed herein (e.g., an extended-release gel formulation containing a biocompatible polymer and an ANG-(1-7) oligopeptide or a variant thereof). The formulations disclosed herein are combined with pharmaceutically acceptable extended-release matrices, such as, e.g., biodegradable polymers (e.g., PLA or PLGA), to form pharmaceutical compositions. “Pharmaceutically” or “pharmaceutically acceptable” refer to molecular entities and compositions that do not produce an adverse, allergic, or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The pharmaceutical compositions disclosed herein may be formulated for intramuscular, subcutaneous, intracerebral (e.g., intraparenchymal or intracerebroventricular), intravenous, transdermal, local, oral, sublingual, subcutaneous, or rectal administration. The active component of the composition (e.g., a ANG-(1-7) oligopeptide of the disclosure or a variant thereof), alone or in combination with another therapeutic agent, can be administered in a unit administration form as a mixture with conventional pharmaceutical supports to subjects in need thereof. Suitable unit administration forms include oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, intracerebral, stereotactic, intranasal administration forms, and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium, or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions, formulations including sesame oil, peanut oil, or aqueous propylene glycol, and sterile powders for the extemporaneous preparation of sterile injectable solutions or dispersions. In all cases, the form must be sterile and must be fluid to the extent that easy syringability is facilitated. It must be stable under the conditions of manufacture and storage and must be preserved against the contaminating action of microorganisms, such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can also be a solvent or dispersion medium containing, e.g., water, ethanol, polyol (e.g., glycerol, propylene glycol, liquid polyethylene glycol, and the like), suitable mixtures thereof, and vegetables oils. The proper fluidity can be maintained by the use of a coating (e.g., lecithin), by the maintenance of the required particle size in the case of dispersion, and by the use of surfactants.

The prevention of the action of microorganisms can be brought about by various antibacterial and antifungal agents such as, e.g., parabens, chlorobutanol, phenol, sorbic acid, thimerosal, and the like. In some cases, it will be pharmaceutical compositions of the disclosure may include isotonic agents such as, e.g., sugars or sodium chloride. Prolonged absorption of the injectable compositions can be brought about by the use of agents delaying absorption such as, e.g., e.g., aluminum monostearate and gelatin. Sterile injectable solutions are prepared by incorporating the active agents in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required additional ingredients from those disclosed herein. In the case of sterile powders for the preparation of sterile injectable solutions, the preferred methods of preparation may be vacuum-drying and freeze-drying techniques which yield a powder of the active ingredient plus any additional desired ingredient from a previously sterile-filtered solution thereof.

Upon formulation, solutions will be administered in a manner compatible with the dosage requirement and in such amount as is therapeutically effective. The formulations are easily administered in a variety of dosage forms, such as the type of injectable solutions described above. For parenteral administration in an aqueous solution, e.g., the solution should be suitably buffered if necessary and the liquid diluent first rendered isotonic with sufficient saline or glucose. These particular aqueous solutions are especially suitable for intravenous, intramuscular, subcutaneous, and intraperitoneal administration. Sterile aqueous media which can be employed are well-known in the art. E.g., one dosage could be dissolved in 1 mL of isotonic NaCl solution and added to 1 L of hypodermoclysis fluid or injected at the proposed site of infusion. Some variation in dosage will necessarily occur depending on the condition of the subject being treated. The practitioner responsible for administration can, in any event, using appropriate patient information and art-recognized methods, determine the appropriate dose for the individual subject.

### Methods of Treatment

### Selection of Subjects

Subjects that may be treated using the compositions and methods described herein are subjects diagnosed as having or at risk of developing vascular dementia.

### Vascular Dementia

Vascular dementia generally refers to dementia caused by insufficient supply of blood to the brain, typically resulting from a series of minor ischemic or hemorrhagic strokes, that eventually leads to lesions of central nervous system (CNS) tissue and cognitive decline. Patients suffering from vascular dementia often experience cognitive decline, motor deficits, abnormal behavior, dysregulated affect, hemiparesis, bradykinesia, hyperreflexia, extensor plantar reflexes, ataxia, pseudobulbar palsy, gait problems, and swallowing difficulties. Generally, vascular dementia can result from one or more minor strokes in different parts of the CNS, including anterior cerebral artery territory, parietal lobes, cingulate gyrus, hippocampus, and/or thalamus.

A subject (e.g., a human) can be diagnosed as having or at risk of developing a vascular dementia on the basis of symptoms listed in the Diagnostic and Statistical Manual of Mental Disorders (DSM-IV), the International Classification of Diseases, Tenth Edition (ICD-10), the National Institute of Neurological Disorders and Stroke, among others. E.g., a subject may be diagnosed as having a vascular dementia on the basis of the presence of one or more of the aforementioned cognitive impairments, as well as one more lesions in the brain and associated blood vessels. Presence of insoluble lipid deposits and clotted blood in the brain may also be indicative of a positive diagnosis of vascular dementia. Noticeable white matter damage occurs in vascular dementia; therefore, white matter atrophy in the brain of an afflicted subject may be indicative of a positive diagnosis for vascular dementia. Current treatment modalities for vascular dementia are largely palliative and do not address the etiological causes of the disease. Therefore, there exists a need for a curative therapy for the treatment and prevention of vascular dementia. The present disclosure provides formulations and compositions containing the same for the treatment and prevention of vascular dementia.

### Routes of Administration

The compositions disclosed herein may be administered to the subject by subcutaneous injection or intramuscular injection. In some embodiments, the compositions disclosed herein are administered to the subject by way of subcutaneous injection. In some embodiments, the compositions disclosed herein are administered to the subject by way of intramuscular injection. The most suitable route for administration in any given case will depend on the particular composition administered, the subject, pharmaceutical formulation methods, administration methods (e.g., administration time and administration route), the subject's age, body weight, sex, severity of the diseases being treated, the subjects diet, and the subject's excretion rate. The disclosed compositions may be administered to a subject one or more times (e.g., 1-10 times) per week, month, or year to a subject for treatment of the disclosed diseases or conditions.

### Combination Therapy

The compositions and methods disclosed herein may be administered to the subject in combination with one or more additional therapeutic modalities (e.g., 1, 2, 3, or more additional therapeutic modalities), including other therapeutic agents or physical interventions (e.g., rehabilitation therapy or surgical intervention). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be performed by any appropriate route including, but not limited to, intramuscular routes, subcutaneous routes, oral routes, intravenous routes, local routes, and direct absorption through mucous membrane tissues. The therapeutic agents can be administered by the same route or by different routes. E.g., a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In cases in which the subject is diagnosed as having or at risk of developing vascular dementia, the second therapeutic agent may include a hypotensive agent, a cholesterol-reducing agent, anti-coagulant agent, anti-platelet agent, thrombolytic agent, insulin, neuroprotective agent, an agent that promotes neuronal axon sprouting, an agent that promotes neuronal regeneration, stem cell therapy, hyperbaric oxygen therapy, and the second therapeutic modality can include surgical intervention or physical rehabilitation.

Non-limiting examples of an anticoagulant agent include coumarins (e.g., warfarin, brodifacoum, or difenacoum), heparin and its derivatives (e.g., low molecular weight heparin), synthetic pentasaccharide inhibitors of factor Xa (e.g., fondaparinux, idraparinux, or idrabiotaparinux), directly acting oral anticoagulants (e.g., dabigatran, rivaroxaban, apixaban, edoxaban, or betrixaban), direct thrombin inhibitors (e.g., hirudin, lepirudin, bivalirudin, argatroban, or dabigatran), anti-thrombin, and other anticoagulants such as batroxobin, hementin, and vitamin E.

Antiplatelet agent that can be used in combination with the compositions and methods disclosed herein include but are not limited to aspirin, triflusal, adenosine diphosphate receptor inhibitors (e.g., cangrelor, clopidogrel, prasugrel, ticagrelor, and ticlopidine), phosphodiesterase inhibitors (e.g., cilostazol), protease-activated receptor-1 antagonists (e.g., vorapaxar), glycoprotein IIB/IIIA inhibitors (e.g., abciximab, eptifibatide, and tirofiban), adenosine reuptake inhibitors (e.g., dipyridamole), and thromboxane inhibitors (e.g., thromboxane synthase inhibitors or thromboxane receptor antagonists (e.g., terutroban)).

Thrombolytic agents suitable for use with the disclosed compositions and methods include but are not limited to alteplase, urokinase, anistreplase, reteplase, streptokinase, kabikinase, tissue plasminogen activator, tenecteplase, and rokinase.

Cholesterol-lowering agents that are suitable for use with the compositions and methods disclosed herein include statins (e.g., atorvastatin, fluvastatin, lovastatin, pitavastatin, pravastatin, rosuvastatin calcium, and simvastatin), proprotein convertase subtilisin/kexin type 9 (PCSK9) inhibitors (e.g., evolocumab and alirocumab), selective cholesterol absorption inhibitors (e.g., ezetimibe), resins (e.g., cholestyramine, colestipol, and colesevelam), and lipid-lowering therapies (e.g., fibrates (e.g., gemfibrozil, fenofibrate, and clofibrate), niacin, omega-3 fatty acid ethyl esters (e.g., Lovaza and Vascepa), and marine-derived omega-3 polyunsaturated fatty acids).

Hypotensive agents that may be used in conjunction with the disclosed compositions and methods include midodrine, Levophed, norepinephrine, phenylephrine, Orvaten, Northera, ephedrine, Vazculep, droxidopa, Akovaz, Biorphen, valsartan, losartan, eprosaran, telmisartan, perindopril, and Corphedra.

Neuroprotective agents that may be used in combination with the disclosed compositions and methods include but are not limited to gangliosides, topiramate, riluzole, methylprednisolone, rivstigmine, selegiline, cilostazol, rasagiline, tenocyclidine, 7-nitroindazole, N-(3-propylcarbamoyloxirane-2-carbonyl)-isoleucyl-proline, huperzine A, SGS-742, D-JNKI-1, nalmefene, ziconotide, dexanabinol, remacemide, clomethiazole, propentofylline, Z-Val-Ala-Asp fluoromethyl ketone, piracetam, epigallocatechin gallate, vinpocetine, tempol, butylphthalide, eliprodil, tirilazad, nefiracetam, gacyclidine, nizofenone, meclofenoxate, linopiridine, fosfructose, methylprednisolone hemisuccinate, dextrorphan, ebselen, almitrine, brimapitide, edaravone, edaravone, minocycline, epoetin-β, trafermin, filgrastim, eicosapentaenoic acid, and pioglitazone.

Agents that promote neuronal axon sprouting and/or neuronal regeneration that may be suitable for use with the compositions and methods disclosed herein include non-steroidal inflammatory drugs (e.g., ibuprofen and indomethacin), inhibitors of protein tyrosine phosphatase sigma, methylprednisolone, GM-1 gangliosides, osteopontin, growth factors minocycline, cAMP modulators, antidepressants, neural stem cells, 4-aminopuridine, and monoclonal antibodies against NI-35 and NOGO.

Surgical intervention may also be used in combination with an extended-release gel formulation of the disclosure for the treatment or prevention vascular dementia in a subject. Non-limiting examples of surgical intervention include surgical resection of the affected CNS region, e.g., endovascular surgery (e.g., mechanical thrombectomy, carotid endarterectomy, angioplasty, coiling, and stents), craniectomy, surgical clipping, stereotactic radiosurgery, and deep brain stimulation. Non-surgical physical interventions including physical therapy, occupational therapy, and recreational therapy may also be administered in combination with the extended-release gel formulation of the disclosure according to the methods disclosed herein.

### Dosing

Subjects that can be treated as described herein are subjects diagnosed as having or at risk of developing vascular dementia. Subjects who can be treated using the disclosed methods and compositions include subjects who have had one or more previous therapeutic interventions related to the treatment of vascular dementia or subjects who have had no previous therapeutic interventions.

Accordingly, the disclosed composition is administered in an amount and for a time effective to result in one of (or more, e.g., 2 or more, 3 or more, 4 or more of): reduction in one or more signs of symptoms of vascular dementia (e.g., amnesia, receptive and/or expressive aphasia, confusion, attention deficit, memory impairment, object recognition impairment, abnormal behaviors such as inappropriate laughing and crying, aggression, loss of impulse control, repetitive speech or actions, mood dysregulation, nausea, loss of consciousness, fatigue, and/or swallowing difficulty are altered or improved in a beneficial manner.

The disclosed formulation or a pharmaceutically acceptable suspension thereof may be administered to a subject (e.g., a human) such that a therapeutically effective amount of the ANG-(1-7) is delivered to the subject, such as, e.g., from about 0.1 to about 1000 mg/day (e.g., 10-100 mg/day, 0.1-50 mg/kg of body weight/day).

### Kits

The disclosure also provides kits that include an extended-release gel formulation or a pharmaceutical composition containing the same for use in the prevention or treatment of vascular dementia. The kits can optionally include an agent or device for delivering the agent to the subject. In other examples, the kits may include one or more sterile applicators, such as syringes or needles. Further, the kits may optionally include other agents, e.g., anesthetics or antibiotics. The kit can also include a package insert that instructs a user of the kit, such as a physician, to perform the methods disclosed herein.

### EXAMPLES

### Example 1: Extended-Release Gel Formulation Containing an Angiotensin-(1-7) Oligopeptide

### Formulation Manufacturing Method

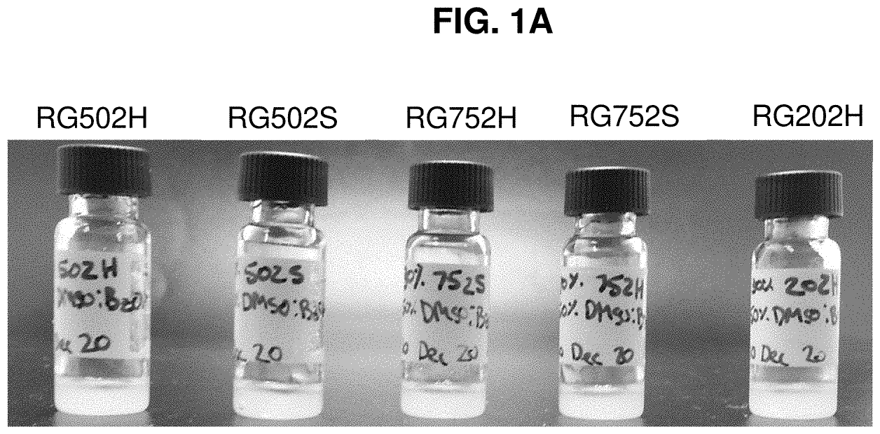

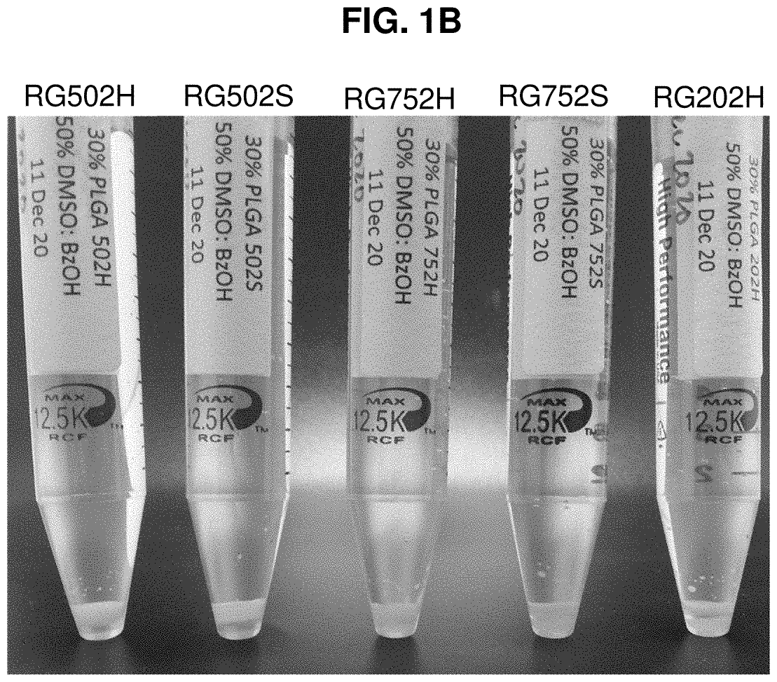

An exemplary extended-release gel formulation containing angiotensin-(1-7)(ANG-(1-7)) oligopeptide was prepared according to the following method. ANG-(1-7) oligopeptide was weighed into a vial and dissolved in organic solvent (e.g., DMSO, NMP, BB, and/or BzOH) and warmed to 37° C. as needed to fully solubilize. Upon dissolving the ANG-(1-7) oligopeptide in solvent, the solution changed appearance from clear to hazy one day after mixing. If an excipient was added, it was mixed into the dissolved ANG-(1-7) solution and left at 37° C. to equilibrate for 30-60 minutes before the PLGA was added. Solid PLGA was weighed and added to the vial (not stirred in) and left at 37° C. until it was wet into the solution (16-24 hours). PLGA solutions were clear before combining with ANG-(1-7). The solution containing the gel and peptide was hazy and viscous after the dissolved peptide and dissolved PLGA solutions were mixed (FIGS. 1A and 1B).

Exemplary formulation compositions containing the ANG-(1-7) oligopeptide in PLA/PLGA polymer formulations are provided in Tables 3 and 4, as is shown below.

TABLE 3
Exemplary formulation compositions containing 20% (w/w) ANG-(1-7) oligopeptide

| | | NMP, | | ANG-(1-7) | Total |
|---|---|---|---|---|---|
| Form | Polymer | DMA or | Polymer | peptide | weight |
| ID | type | DMSO (mg) | amount (mg) | amount (mg) | (mg) |
| A1 | RG502H | 125 | 75 | 50 | 250 |
| B1 | RG752H | 125 | 75 | 50 | 250 |
| C1 | RG503H | 125 | 75 | 50 | 250 |
| D1 | RG202H | 125 | 75 | 50 | 250 |

TABLE 4
Exemplary formulation compositions containing 13% (w/w) ANG-(1-7) oligopeptide

| | | NMP, | | ANG-(1-7) | Total |
|---|---|---|---|---|---|
| Form | Polymer | DMA or | Polymer | peptide | weight |
| ID | type | DMSO (mg) | amount (mg) | amount (mg) | (mg) |
| A1 | RG752H | 250 | 187.5 | 62.5 | 500 |
| B1 | RG752S | 250 | 187.5 | 62.5 | 500 |
| C1 | RG202H | 250 | 187.5 | 62.5 | 500 |

ANG-(1-7) gel formulation compositions were also produced in combination with various hydrophobic excipients, including the hydrophobic carboxylic acid, oleic acid. Exemplary formulations containing 17% (w/w) ANG-(1-7) are described in Table 5, as is shown below.

TABLE 5
Exemplary formulation compositions containing hydrophobic excipients

| | Polymer | ANG-(1-7) | DMSO | Excipient | PLGA |
|---|---|---|---|---|---|
| Solvent excipient | type | peptide (mg) | (mg) | (mg) | (mg) |
| DMSO: Oleic Acid | RG752S | 166.8 | 400 | 100 | 333.2 |
| DMSO: Olive Oil | RG752S | 166.8 | 400 | 100 | 333.2 |
| DMSO: Octanoic Acid | RG752S | 166.8 | 400 | 100 | 333.2 |
| DMSO: Ethyl Oleate | RG752S | 166.8 | 400 | 100 | 333.2 |
| DMSO: Ethyl Laurate | RG752S | 166.8 | 400 | 100 | 333.2 |
| DMSO: Propylene Glycol Dioleate | RG752S | 166.8 | 400 | 100 | 333.2 |
| DMSO: Glyceryl MonoOleate | RG752S | 166.8 | 400 | 100 | 333.2 |
| DMSO: Palmitic Acid | RG752S | 41.7 | 101.2 | 23.8 | 83.3 |
| DMSO: Myristic Acid | RG752S | 41.7 | 103.1 | 21.9 | 83.3 |
| DMSO: Sodium Stearate | RG752S | 41.7 | 96.6 | 28.4 | 83.3 |

### Solubility Assessment

A small sample containing the (co-)polymer of interest (e.g., PLA or PLGA) and the ANG-(1-7) oligopeptide (total sample size: ˜1 mg) was placed into a microfuge tube. The solvent of interest (e.g., DMSO, NMP, BB, and/or BzOH) was added in μL aliquots until the drug compound was in solution. The samples were left overnight to fully dissolve and were sonicated the following day, as was necessary to fully solubilize the sample. Solubility was determined when/if a clear solution was obtained.

Solubility assessment was performed for the ANG-(1-7) oligopeptide of the disclosure across multiple different solvents shown in Table 6, below. The PBS and DMSO samples showed very high solubility and did not require sonication. All other solvents required sonication to fully dissolve the ANG-(1-7) oligopeptide. DMSO is a suitable starting solvent for gel formulation evaluation.

TABLE 6
Solubility assessment of ANG-(1-7) in various solvents

| Solvent | Concentration | Appearance at 0-24 hours |
|---|---|---|
| DMSO | 500 mg/mL | Clear colorless, fluid |
| PBS at pH 7.4 | 500 mg/mL | Clear colorless, fluid |
| Oleic Acid | 250 mg/mL | Translucent white, viscous |
| Ethoxylated Castor Oil | 250 mg/mL | Cloudy, gel |
| Ethyl Oleate | 250 mg/mL | Cloudy, fluid |
| Triacetin | 250 mg/mL | Cloudy, viscous |
| Ethyl Laureate | 250 mg/mL | Cloudy, fluid |
| Triethyl Citrate | 250 mg/mL | Cloudy, viscous |
| Polyethylene Glycol 300 | 250 mg/mL | Translucent white, viscous |
| NMP | 250 mg/mL | Clear colorless, fluid |
| Benzyl Alcohol | 250 mg/mL | Translucent white, fluid |
| Benzyl Benzoate | 250 mg/mL | Cloudy, fluid |

### Determination of Drug Concentration in the Manufactured Formulation

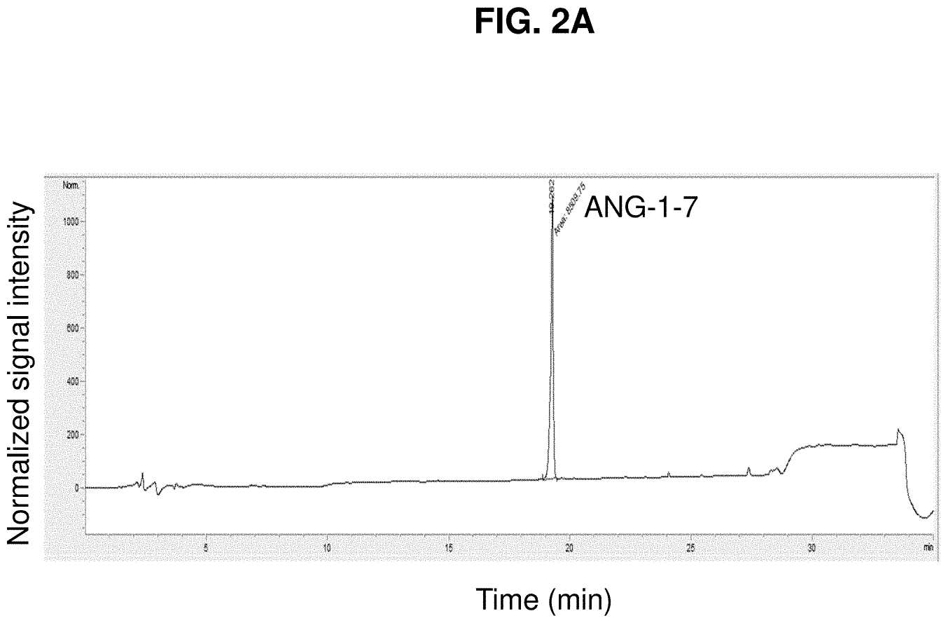

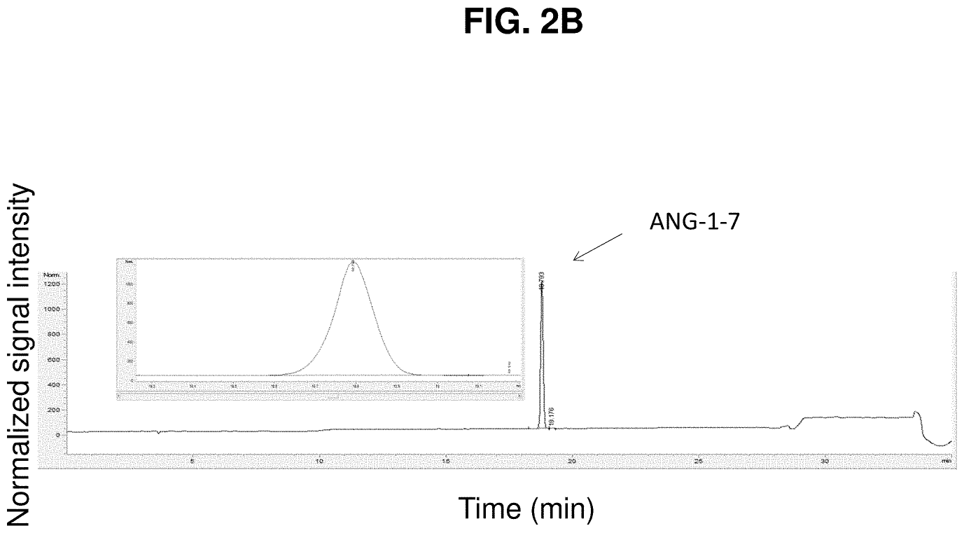

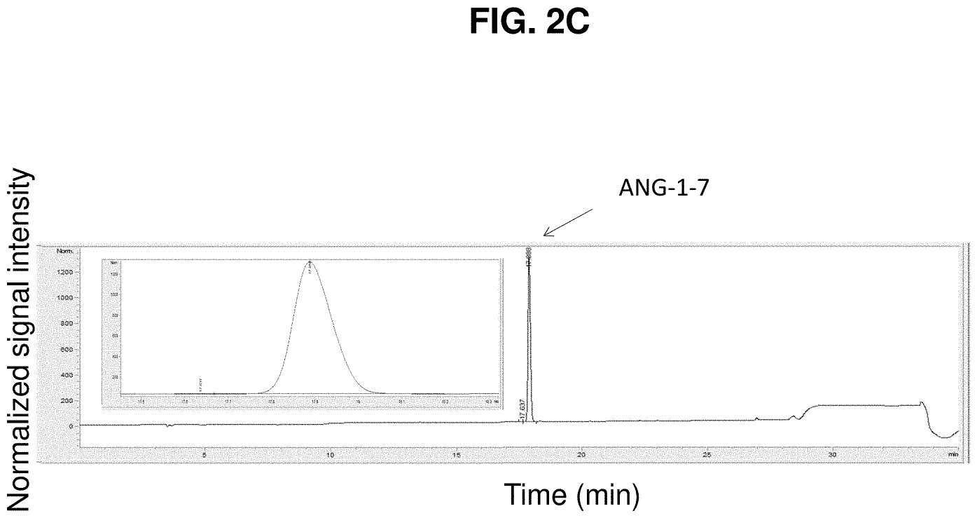

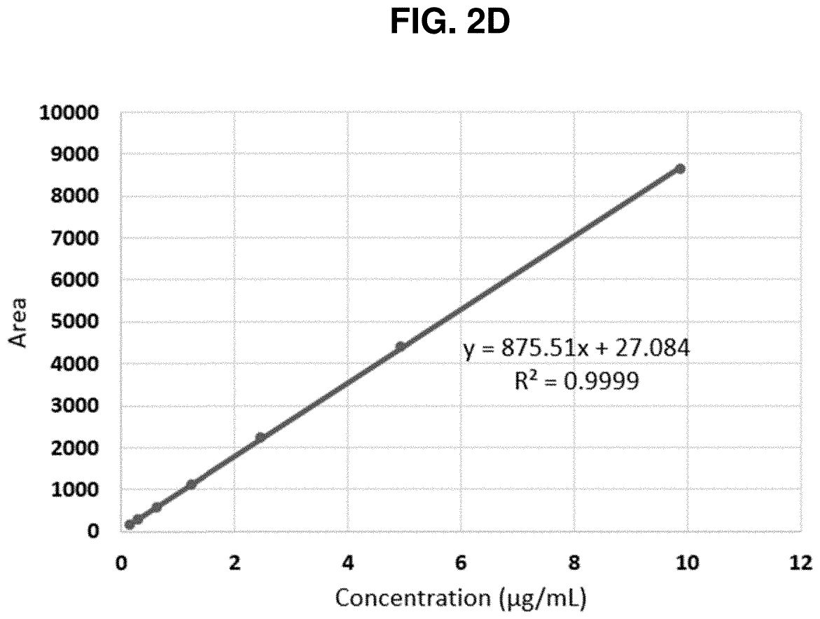

The manufactured gel formulation containing the ANG-(1-7) oligopeptide was mixed thoroughly with a pipette before 30 μL was pipetted into an Eppendorf tube. The gel in the tube was dissolved in 100 μL of NMP by sonicating for 2-5 minutes. After 200 μL of 0.1% TFA in water was added to the tube, it was vortexed to precipitate the PLGA from the formulation. The tube was centrifuged at 12,000 rpm for 10 minutes to force the precipitate to bottom of tube. 20 μL of supernatant was removed and the remaining mixture was diluted in 580 μL water. The sample was subsequently run through a reverse-phase high-performance liquid chromatography (RP-HPLC) column using the conditions shown in Tables 7 and 8. Core loading was done in triplicate to ensure sample homogeneity. Retention times for ANG-(1-7) peptide gel-formulations are shown for three different operating temperatures, namely 25° C., 40° C., and 60° C., in FIGS. 2A-2C, respectively. The area under the elution curve of had a strong linear correlation with the concentration of ANG-(1-7) peptide in the tested formulation (FIG. 2D), suggesting that RP-HPLC is an effective method for determining the concentration and purity of ANG-(1-7) oligopeptides in the gel formulations of the disclosure.

TABLE 7
HPLC conditions for measuring drug concentration

| | |
|---|---|
| RP-HPLC | Agilent 1100 with diode array detector (DAD) |
| Column and | Waters X-Bridge C18 column |
| Guard Cartridge: | 250 × 4.6 mm, 3.5 μm |
| | particle size, 133 Å pore size |
| Auto sampler Temperature: | Ambient |
| Column Temperature: | 25 ± 2° C. |
| Injection Volume: | 10 μL |
| UV Detection: | 220 nm and 280 nm |
| Flow Rate: | 1 mL/min |
| Mobile Phase A: | 0.1% Trifluoroacetic Acid (TFA) in Water |
| Mobile Phase B: | 0.1% TFA in Acetonitrile (ACN) |

TABLE 8
Solvent gradient for HPLC elution

| | 0.1% | 0.1% |
|---|---|---|
| | TFA in | TFA in |
| Time (Min) | Water (%) | ACN (%) |
| 0.0 | 95 | 5 |
| 5.0 | 95 | 5 |
| 25.0 | 55 | 45 |
| 25.10 | 10 | 90 |
| 30.0 | 10 | 90 |
| 30.10 | 95 | 5 |
| 35.0 | 95 | 5 |

### In Vitro Release

The gel formulation was mixed thoroughly with a pipette before 100 μL was pipetted into a 15 mL conical tube. 10 mL of PBS release media was added to the tube and kept on a 37° C. shaker table to incubate. Sampling was performed 3-4 times during the first day and every day for 7 days, and once a week thereafter before final mass balance was determined. At every sampling timepoint, the tube was centrifuged for 10 minutes at 3000 rpm to force the gel pellet to the bottom of the tube. The supernatant was analyzed by RP-HPLC for measuring the concentration of released active pharmaceutical ingredient (API), e.g., ANG-(1-7) oligopeptide. After at every timepoint that sample was removed, complete in vitro PBS release media was replenished. In vitro release was subsequently plotted as cumulative release over time. The gel polymers tested included 50% acid-capped PLA and 50% acid-capped PGA (RG502H), 50% ester-capped PLA and 50% ester-capped PGA (RG502S), 75% acid-capped PLA and 25% acid-capped PGA (RG752H), 75% ester-capped PLA and 25% ester-capped PGA (RG752S), and 100% acid-capped PLA (RG202H).

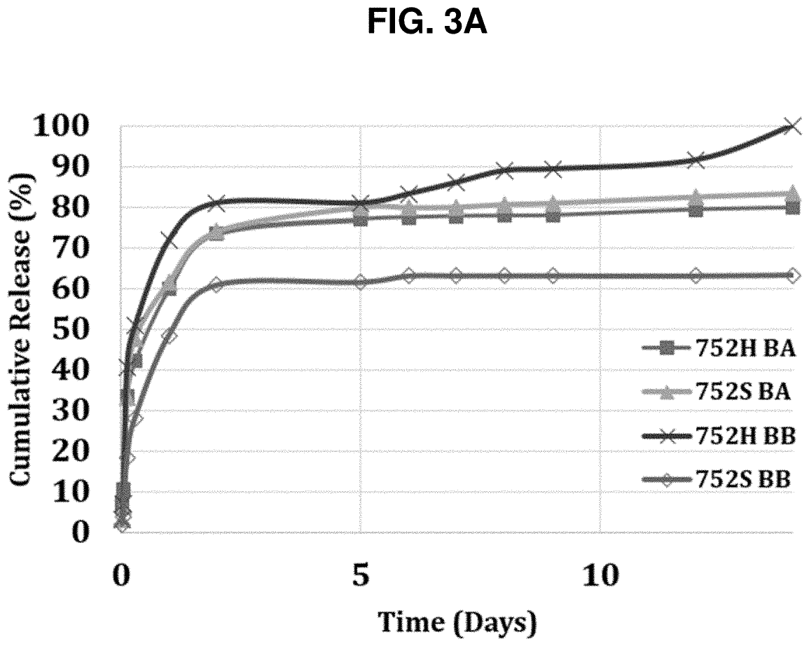

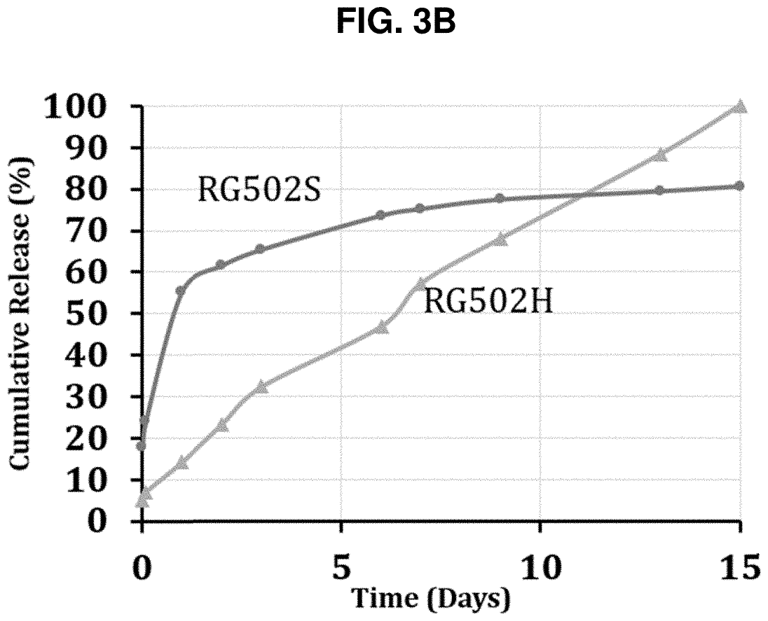

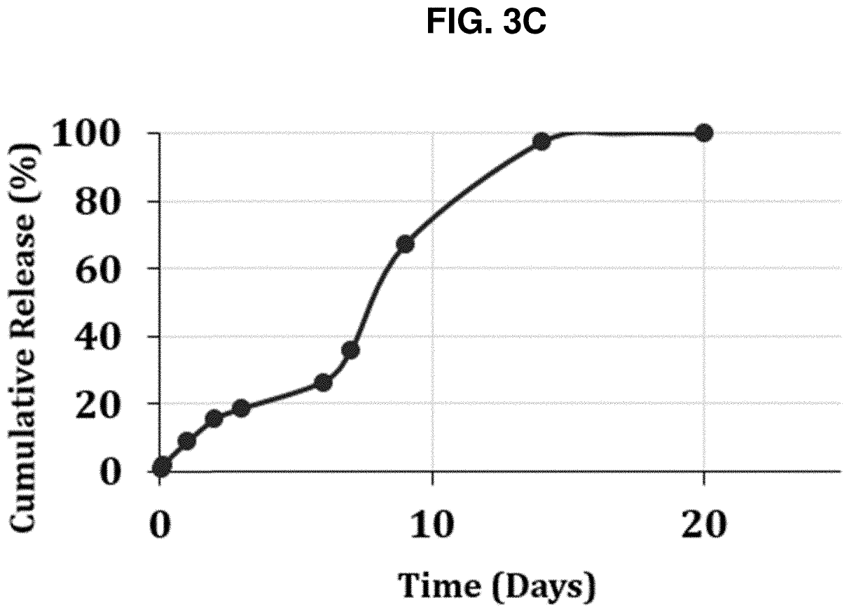

The various tested ANG-(1-7) peptide formulations, namely RG502H, RG502S, RG752H, RG752S, and RG202H, exhibited different release kinetics suitable for distinct administration regimens. In particular, some formulations exhibited release profiles that render them suitable for a once-weekly administration regimen. Three polymer formulations of ANG-(1-7) showed in vitro release (IVR) profiles that range from a couple of days to one week (RG752H and RG752S). The formulations contained 37% PLGA by weight. The solvent used in the formulation was 75% DMSO with either 25% benzyl alcohol (BA) or 25% benzyl benzoate (BB)(FIG. 3A). IVR profile for the short-acting RG502H and RG502S polymer formulations of ANG-(1-7) showed release profiles that range from a one to two weeks. The formulations contained 13% of ANG-(1-7) and 37% PLGA by weight. The solvent used in the formulation was DMSO containing two equivalents of oleic acid (molar equivalents to ANG-(1-7))(FIG. 3B). IVR profile for the short-acting RG502H polymer formulations of ANG-(1-7) showed release profiles that range from a one to two weeks. The solvent used in the formulation was DMSO containing four equivalents of oleic acid (molar equivalents to ANG-(1-7))(FIG. 3C).

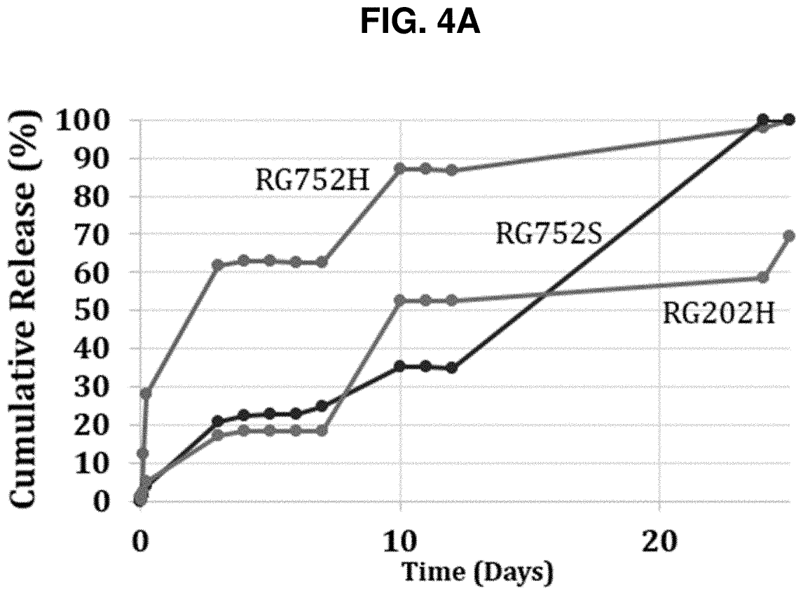

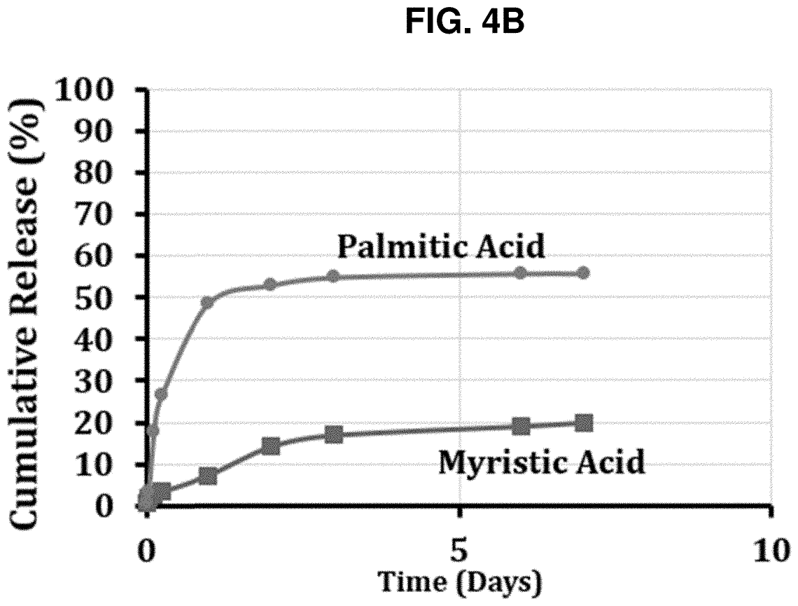

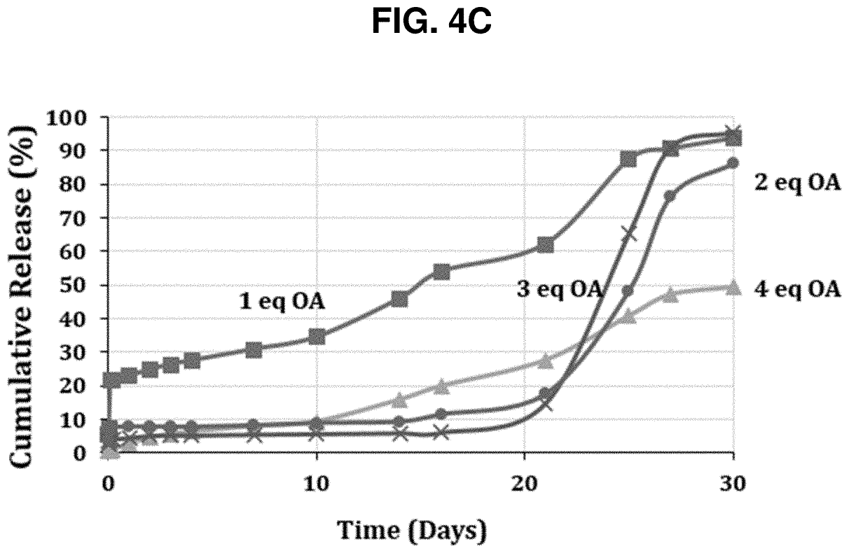

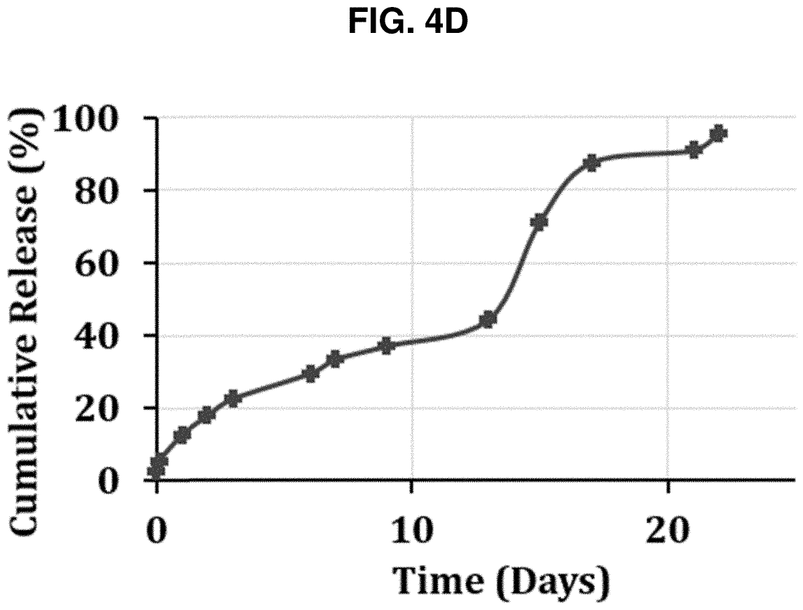

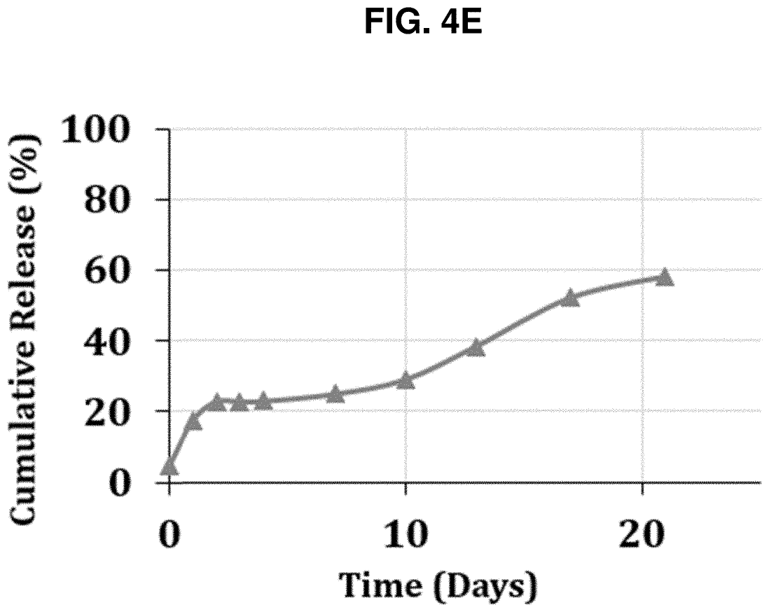

Other formulations exhibited release profiles that render them suitable for a once-monthly administration regimen. In particular, three polymer formulations of ANG-(1-7) showed release profiles that range from two weeks to one month release (RG752H, RG752S, and RG202H)(FIG. 4A). IVR profile for the polymer formulation, RG752S, of ANG-(1-7) showed release profiles that range from two weeks to one month release. These formulation compositions contained 2 equivalents (molar ratio to ANG-(1-7)) of the excipients myristic acid or palmitic acid in the DMSO solution (FIG. 4B). IVR profile for the hydrophobic polymer formulation, RG752S, of ANG-(1-7) containing various equivalents (eq) of oleic acid (OA; molar equivalents to ANG-(1-7)) in DMSO solution. These formulations showed a two to four-week IVR profile (FIG. 4C). IVR profile for the polymer formulation, RG752H, of ANG-(1-7) containing two equivalents of OA (molar equivalents to ANG-(1-7)) in DMSO solution. This formulation showed a two to four-week IVR profile (FIG. 4D). IVR profile for the polymer formulation, RG752H, of ANG-(1-7) containing four equivalents of OA (molar equivalents to ANG-(1-7)) in the DMSO solution. This formulation showed a two to four-week IVR profile (FIG. 4E). Overall, the ester-capped (S) polymer formulations exhibited a slower IVR as compared to acid-capped (H) polymer formulations.

Therefore, the disclosed gel formulations containing ANG-(1-7) may be manufactured according to the methods described herein in order to provide therapeutically-beneficial ANG-(1-7) extended-release gel formulations having one-week or one-month release kinetics.

### Example 2: Extended-Release Gel Formulation Containing an Angiotensin-(1-7) Oligopeptide Variant, PNA5

A sustained release gel formulation containing a variant ANG-(1-7) oligopeptide, namely PNA5 (SEQ ID NO: 10), was prepared according to the formulation manufacturing methods described in Example 1, above.

### Solubility Assessment

Solubility of PNA5 in various solvents was tested and for potential use with this peptide. Table 9, below, shows solubility assessment of PNA5 in various solvents. Similar to ANG-(1-7), PNA5 exhibited high solubility in DMSO and PBS and did not require sonication in these two solvents to become fully soluble. All other solvents required sonication to fully dissolve PNA5. DMSO is a suitable starting solvent for gel formulation evaluation using the PNA5 peptide. Benzyl benzoate and benzyl alcohol may also be useful solvents or solvent additives.

TABLE 9
Solubility assessment of PNA5 oligopeptide in various solvents

| | | Observation |
|---|---|---|
| Solvent | Concentration | at 0-24 hours |
| DMSO | 500 mg/mL | Clear colorless, fluid |
| PBS | 100 mg/mL | Clear colorless, fluid |
| Oleic Acid | 100 mg/mL | Insoluble |
| Ethoxylated Castor Oil | 100 mg/mL | Insoluble |
| Ethyl Oleate | 100 mg/mL | Insoluble |
| Triacetin | 100 mg/mL | Insoluble |
| Ethyl Laurate | 100 mg/mL | Insoluble |
| Triethyl Citrate | 100 mg/mL | Insoluble |
| Polyethylene Glycol 300 | 100 mg/mL | Insoluble |
| NMP | 100 mg/mL | Insoluble |
| Benzyl Alcohol (BA) | 100 mg/mL | Clear colorless, fluid |
| Benzyl Benzoate (BB) | 100 mg/mL | Clear colorless, fluid |
| DMA | 100 mg/mL | Insoluble |

Exemplary sustained release formulations containing PNA5 (14% (w/w)) in various solvents/solvent combinations and, in some cases, including oleic acid as an excipient are shown in Table 10, below.

TABLE 10
Exemplary PNA5 sustained release gel formulation compositions

| | | | | Oleic | |
|---|---|---|---|---|---|
| | | PNA5 | Solvent | Acid | PLGA |
| Solvent | PLGA | (mg) | (mg) | (mg) | (mg) |
| DMSO | RG502S | 83.4 | 250 | 0 | 166.6 |
| DMSO | RG752H | 83.4 | 250 | 0 | 166.6 |
| DMSO | RG752S | 83.4 | 250 | 0 | 166.6 |
| 75:25 | RG752S | 83.4 | 187.5 DMSO | 0 | 166.6 |
| DMSO:BB | | | 62.5 BB | | |
| DMSO:BB:OA | RG752S | 83.4 | 153.7 DMSO | 45 | 166.6 |
| | | | 51.3 BB | | |
| DMSO:OA | RG752S | 83.4 | 205 | 45 | 166.6 |

### In Vitro Release

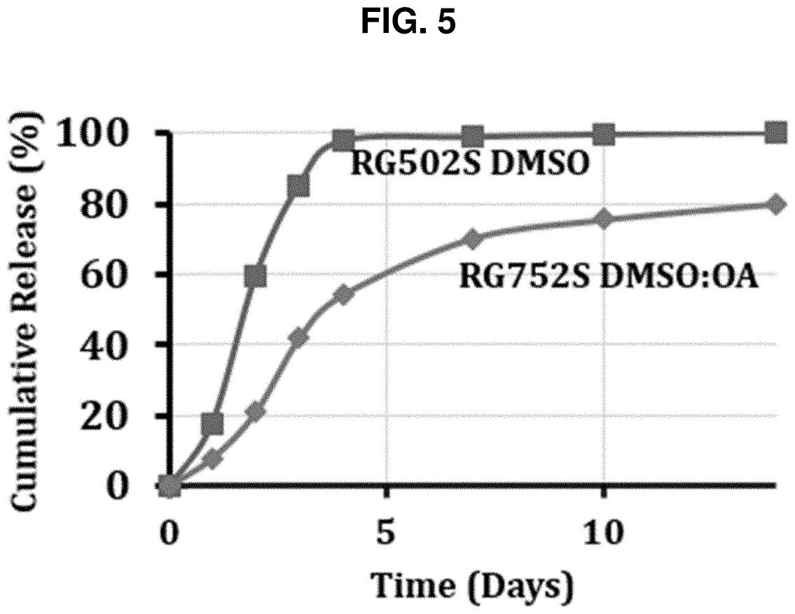

In vitro release properties of various PNA5 sustained release gel formulations were tested according to the methods described in Example 1, above. Similar to the ANG-(1-7) gel formulations described in Example 1, some PNA5-based gel formulations exhibited release kinetics that would make them suitable for a once-weekly or a once-monthly administration regimen. For example, IVR profile for the polymer formulation, RG502S, of PNA5 in DMSO solution showed a one-week IVR profile. IVR profile for the polymer formulation RG752S in DMSO solution containing 45 mg OA showed a one to two-week release profile (FIG. 5).

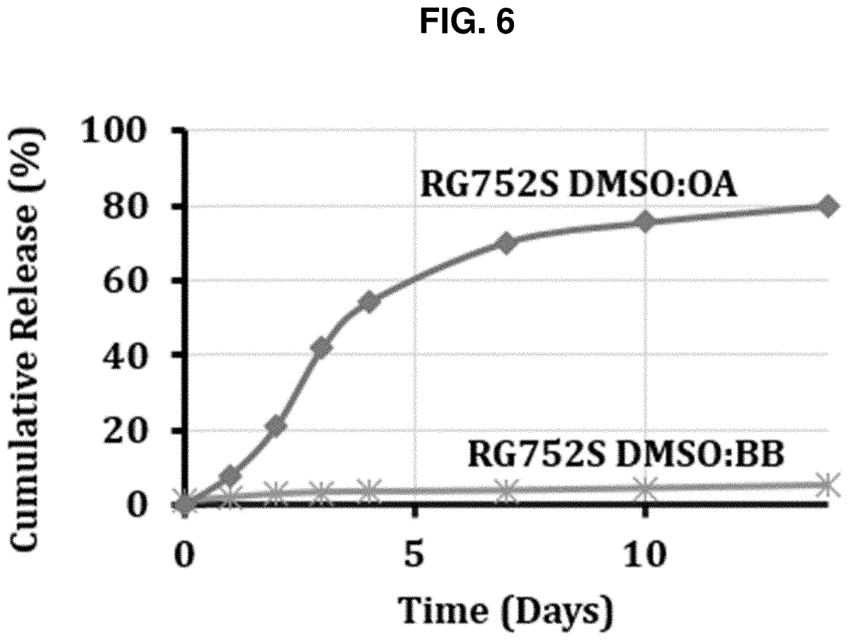

IVR profile for the polymer formulation RG752S in DMSO solution containing 45 mg OA showed a two-week release profile. IVR profile for the polymer formulation RG752S in DMSO solution containing 25% BB showed a very long release profile of at least four weeks (FIG. 6).

Therefore, the disclosed gel formulations containing PNA5 may be manufactured according to the methods described herein in order to provide therapeutically-beneficial PNA5 extended-release gel formulations having one-week or one-month release kinetics.

### Example 3: Extended-Release Gel Formulation Containing an Angiotensin-(1-7) Oligopeptide Characterization In Vivo

Male and female Sprague-Dawley rats with average weight of 200-224 g were used for the in vivo study. Animals were maintained in a 12:12 light-dark cycle with free access to food with all testing and procedures performed during the light cycle. The experimental procedures described in this study received prior approval from the Institutional Animal Care and Use Committee and were in compliance with the guide for the Care and Use of Laboratory Animals published by the US National Institutes of Health (NIH Publication No. 85-23, revised 1996). Animals were divided into seven groups and administered either Formulation 1,2,3,4,5,6 or 7 as described in Table 11. The total volume for each formulation was 250 μL and was administered via a single subcutaneous injection. Immediately following injection, blood samples were collected using a chronically implanted jugular catheter. At each time point approximately 200 μL was collected into centrifuge tubes pre-treated with a protease inhibitor cocktail. The collected blood was collected was immediately centrifuged at 4300 g at 8∘° C. for 15 minutes. The separated plasma samples were frozen at −80∘° C. until analysis. Blood samples (0.5 mL) were collected at 5 minutes and 30 min, then after 1, 2, 4, 8 and 24 hours and then at days 4, 7, 10, 21, 28, 35, 42, 49, and 56 days post-injection. The plasma concentration of the drug in each sample was measured using HPLC-MS. The LC-MS method used as the basis for this analysis was fully validated to CLIA standards for use as a clinical diagnostic test. Prior to each batch, a 12-point calibration curve (4.88 ng/ml to 100000 ng/ml) and 3 QC replicates (1000 ng/ml) were run to evaluate the limits of detection, accuracy, and reproducibility. All samples were run in duplicate. The results of this experiment are described in Table 12.

TABLE 11
Formulations containing an angiotensin-(1-7) oligopeptide characterized in vivo

| Formulation | | | | | % | % |
|---|---|---|---|---|---|---|
| # | API | Conc. | Solvent | PLGA | Solvent | PLGA |
| 1 | Ang 1-7 | 60 mg/mL | DMSO | 502H | 59% | 35% |
| 2 | Ang 1-7 | 60 mg/mL | DMSO | 502H | 59% | 35% |
| 3 | Ang 1-7 | 120 mg/mL | DMSO | 502H | 53% | 35% |
| 4 | PNA-5 | 50 mg/mL | 3:2 DMSO:BB | 752S | 55% | 40% |
| 5 | PNA-5 | 50 mg/mL | 3:2 DMSO:BB | 502H | 55% | 40% |
| 6 | PNA-5 | 50 mg/mL | 2:3 DMSO:BB | 502H | 60% | 35% |
| 7 | PNA-5 | 50 mg/mL | 1:1 NMP:BB:MA | 502H | 65% | 30% |

TABLE 12
Pharmacokinetic results for angiotensin-(1-7) formulations administered in vivo

| | Formulation | Formulation | Formulation | Formulation | Formulation | Formulation | Formulation |
|---|---|---|---|---|---|---|---|
| PK values | 1 | 2 | 3 | 4 | 5 | 6 | 7 |
| t½ | 16.0 | 7.5 | 23.6 | 15.3 | 19.6 | 20.6 | 27.2 |
| Tmax | 0.0 | 8.0 | 0.0 | 0.0 | 0.0 | 0.0 | 0.2 |
| Cmax | 170.4 | 38.9 | 695.4 | 73.7 | 60.0 | 55.8 | 134.3 |
| (ng/ml) | | | | | | | |
| Tlag | 0.0 | 0.0 | 0.0 | 0.0 | 0.0 | 0.0 | 0.0 |
| Clast_obs/ | 0.0 | 0.0 | 0.0 | 0.0 | 0.0 | 0.1 | 0.0 |
| Cmax | | | | | | | |
| AUC 0-t | 41.1 | 15.0 | 38.0 | 87.8 | 177.1 | 176.0 | 177.6 |
| (ng/ml*day) | | | | | | | |
| AUC 0- | 45.8 | 15.5 | 42.7 | 143.5 | 238.4 | 296.9 | 313.0 |
| inf_obs | | | | | | | |
| (ngml*day) | | | | | | | |

### Example 4: Assessment of Protection of Cognitive Function In Vivo

The Novel Object Recognition (NOR) task can be used to evaluate cognition, particularly recognition memory, in models of dementia and brain dysfunction. This test is based on the spontaneous tendency of animals and humans to spend more time exploring a novel object than a familiar one. The choice to explore the novel object reflects the use of learning and recognition memory (Ennaceur A and Delacour J (1988) A new one-trial test for neurobiological studies of memory in rats. 1: Behavioral data. Behav Brain Res 31:47-59).

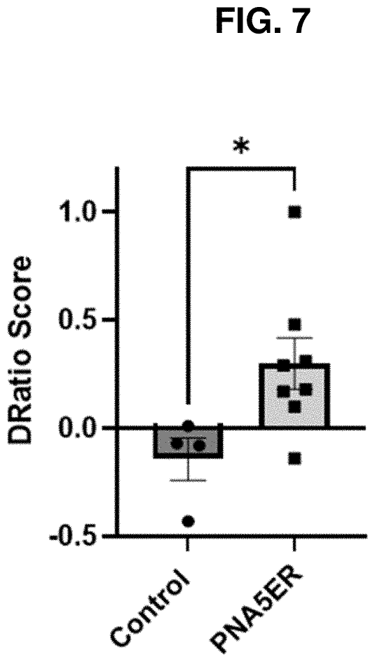

FIG. 7 illustrates the effects of treatment with PNA5-ER on memory as determined in mice by NOR. Animal Groups: A total of 12, male C57BI/6J adult mice (Harlan, 8-10 weeks old) were used. For the HF mice, myocardial infarction (MI) was induced by ligation of the left coronary artery. After 8 weeks post MI, mice were treated with either a single 250 microliter injection of Formulation 4, as described above in Table 11, (50 mg/ml, n=8) or vehicle control (n=4). Cognitive function, as measured by the NOR test, was quantified using the discrimination ratio score. To calculate the discrimination ratios, the time spent exploring the novel object minus time spent exploring the familiar object during the test phase divided by the total exploration time. DRatio=(t novel−t familiar)/(t novel+t familiar). A negative, or zero Discrimination ratio indicates impaired cognitive function. HF mice treated with vehicle control had an average negative Discrimination ratio of −0.14±SE 0.09 that was significantly lower those treated with PNA5ER (0.29±SE 0.11). Students-t test, *=p<0.05.

### Example 5: Treatment of a Human Subject Having or at Risk of developing Vascular Dementia Using an Extended-Release Gel Formulating Containing an Angiotensin-(1-7) Oligopeptide

According to the methods disclosed herein, a physician of skill in the art can treat a subject, such as a human subject diagnosed as having or at risk of developing vascular dementia. The method of treatment can include diagnosing or identifying a patient as a candidate for treatment with the formulation. The formulation can include a biocompatible (co-)polymer (e.g., poly-lactic acid (PLA), poly-(lactic co-glycolic acid)(PLGA), or sucrose acetoisobutyrate (SAIB) polymer, an angiotensin-(1-7) oligopeptide (ANG-(1-7)), and an injection vehicle (e.g., phosphate buffered saline (PBS)) suitable for administration to the subject.

To treat the patient, a physician of skill in the art can administer the formulation to the subject. The formulation is administered by any suitable means including intramuscular injection or subcutaneous injection. The physician of skill in the art may employ a needle size that is appropriate for the syringability of the formulation (e.g., a needle having a diameter of, e.g., 20-25 gauge). The formulation administered such that a therapeutically effective amount of the ANG-(1-7) is delivered to the subject, such as, e.g., from about 0.1 to about 1000 mg/day (e.g., 10-100 mg/day, 0.1-50 mg/kg of body weight/day).

The composition may be administered bimonthly, once a month, once every two weeks, or at least once a week or more (e.g., 1, 2, 3, 4, 5, 6, or 7 times a week or more). In some embodiments, the formulation may be administered in combination with a second therapeutic modality, such as a second therapeutic agent (e.g., second therapeutic agent disclosed herein), surgical intervention (e.g., endovascular surgery, such as mechanical thrombectomy, carotid endarterectomy, angioplasty, coiling, and stents), craniectomy, surgical clipping, ad stereotactic radiosurgery), or non-surgical intervention including physical therapy, occupational therapy, recreational therapy, and speech therapy.

The extended-release gel formulation is administered to the subject in an amount sufficient to decrease one or more of: amnesia, receptive and/or expressive aphasia, confusion, attention deficit, memory impairment, object recognition impairment, abnormal behaviors such as inappropriate laughing and crying, aggression, loss of impulse control, repetitive speech or actions, mood dysregulation, swallowing difficulty. The above-listed symptoms of vascular dementia may be assessed using standard methods, such as neurological examination, blood testing, CT scan, MRI scan, Doppler ultrasound, and arteriography. Measures of symptoms from before and after administration of the composition can be compared to evaluate the efficacy of the treatment. A finding of a reduction in the symptoms of vascular dementia described above indicates that the formulation has successfully treated the vascular dementia in the subject.

### Example 6: Treatment of a Human Subject Having or at Risk of Developing Vascular Dementia Using an Extended-Release Gel Formulating Containing a PNA5 Oligopeptide

According to the methods disclosed herein, a physician of skill in the art can treat a subject, such as a human subject diagnosed as having or at risk of developing vascular dementia. The method of treatment can include diagnosing or identifying a patient as a candidate for treatment with the formulation. The formulation can include a biocompatible PLA polymer, PLGA co-polymer, or SAIB polymer, a variant of ANG-(1-7), namely PNA5, and an injection vehicle (e.g., PBS) suitable for administration to the subject.

To treat the patient, a physician of skill in the art can administer the formulation to the subject. The formulation is administered by any suitable means including intramuscular injection or subcutaneous injection. The physician of skill in the art may employ a needle size that is appropriate for the syringability of the formulation (e.g., a needle having a diameter of, e.g., 20-25 gauge). The formulation administered such that a therapeutically effective amount of the PNA5 is delivered to the subject, such as, e.g., from about 0.1 to about 1000 mg/day (e.g., 10-100 mg/day, 0.1-50 mg/kg of body weight/day).

The composition may be administered bimonthly, once a month, once every two weeks, or at least once a week or more (e.g., 1, 2, 3, 4, 5, 6, or 7 times a week or more). In some embodiments, the formulation may be administered in combination with a second therapeutic modality, such as a second therapeutic agent (e.g., second therapeutic agent disclosed herein), surgical intervention (e.g., endovascular surgery, such as mechanical thrombectomy, carotid endarterectomy, angioplasty, coiling, and stents), craniectomy, surgical clipping, ad stereotactic radiosurgery), or non-surgical intervention including physical therapy, occupational therapy, recreational therapy, and speech therapy.

The extended-release gel formulation is administered to the subject in an amount sufficient to decrease one or more of: amnesia, receptive and/or expressive aphasia, confusion, attention deficit, memory impairment, object recognition impairment, abnormal behaviors such as inappropriate laughing and crying, aggression, loss of impulse control, repetitive speech or actions, mood dysregulation, swallowing difficulty. The above-listed symptoms of vascular dementia may be assessed using standard methods, such as neurological examination, blood testing, CT scan, MRI scan, Doppler ultrasound, and arteriography. Measures of symptoms from before and after administration of the composition can be compared to evaluate the efficacy of the treatment. A finding of a reduction in the symptoms of vascular dementia described above indicates that the formulation has successfully treated the vascular dementia in the subject.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. An extended-release injectable gel formulation comprising at least one biocompatible polymer comprising polylactic acid (PLA) or poly(lactic-co-glycolic acid) (PLGA) having dispersed therein an effective amount of an oligopeptide of SEQ ID NO: 1, wherein the polymer and the oligopeptide are dissolved in an organic solvent, wherein the formulation is configured to have an in vitro release profile comprising a sustained release of at least 60% of the effective amount of the oligopeptide within 48 hours following placement of the formulation in a release medium (t₀) and an initial burst release not greater than 30% of the effective amount of the oligopeptide within 24 hours following t₀, wherein the sustained release does not exceed an average rate of release of the oligopeptide that is greater than 20%/24 hours for a period equal to or greater than seven days following t₀, as measured by high performance liquid chromatography (HPLC) and/or mass spectrometry (MS) at an operating temperature of 37° C., wherein the release medium is phosphate buffered saline (PBS) having a temperature of 37° C. and pH 7.4.

2. An extended-release injectable gel formulation comprising at least one biocompatible polymer comprising PLA or PLGA having dispersed therein an effective amount of an oligopeptide of SEQ ID NO: 1, wherein the polymer and the oligopeptide are dissolved in an organic solvent, wherein the formulation is configured to have an in vitro release profile comprising a sustained release of at least 60% of the effective amount oligopeptide within 25 days following placement of the formulation in a release medium (t₀) and an initial burst release not greater than 30% of the effective amount of the oligopeptide within 24 hours following t₀, wherein the sustained release does not exceed an average rate of release of the oligopeptide that is greater than 30%/168 hours for a period equal to or greater than 30 days following t₀, as measured by HPLC and/or MS at an operating temperature of 37° C., wherein the release medium is PBS having a temperature of 37° C. and pH 7.4.

3-4. (canceled)

5. The formulation of claim 2, wherein the formulation comprises PLA in an amount of 100% of a total number of monomers in the biocompatible polymer and the biocompatible polymer has a molecular weight of between 10,000 and 18,000 Daltons.

6. The formulation of claim 2, wherein the PLGA comprises PLA in an amount of 75% and polyglycolic acid (PGA) in an amount of 25% of a total number of monomers in the biocompatible polymer and the biocompatible polymer has a molecular weight of between 4,000 and 15,000 Daltons.

7. The formulation of claim 2, wherein the PLGA comprises PLA in an amount of 50% and PGA in an amount of 50% of a total number of monomers in the biocompatible polymer and the biocompatible polymer has a molecular weight of between 7,000 and 17,000 Daltons.

8. The formulation of claim 2, wherein the PLGA or PLA is ester-capped or acid-capped.

9. (canceled)

10. The formulation of claim 2, wherein the formulation comprises the biocompatible polymer in an amount of 1-300 mg.

11. The formulation of claim 2, wherein the biocompatible polymer comprises 1-50% (w/w) of a total mass of the formulation.

12. The formulation of claim 2, wherein the organic solvent is dimethyl sulfoxide (DMSO), benzoic acid (BzOH), N-methyl-2-pyrrolidone (NMP), benzyl benzoate (BB) or any combination thereof.

13-14. (canceled)

15. The formulation of claim 2, further comprising a release modifier, optionally wherein the release modifier is selected from the group consisting of a hydrophobic carboxylic acid, oleic acid, palmitic acid, myristic acid, benzyl benzoate, ethoxylated castor oil, palm oil, ethyl oleate, triacetin, ethyl laureate, triethyl citrate, polyethylene glycol 300, dimethylacetamide (DMA), and any combination thereof.

16-17. (canceled)

18. The formulation of claim 2, wherein the effective amount of the oligopeptide is 50 mg.

19. The formulation of claim 2, wherein the formulation comprises the oligopeptide at a concentration of 200 mg/mL and/or the oligopeptide comprises 15-45% (w/w) of a total mass of the formulation.

20. The formulation of claim 2, wherein the formulation comprises the biocompatible polymer and the oligopeptide in a weight ratio of the biocompatible polymer to the oligopeptide of between 1:2 and 1:3.

21. The formulation of claim 2, wherein the formulation is provided in an injectable volume, optionally wherein the injectable volume is 0.5-2 mL, optionally wherein the formulation provides injectability of the formulation into a host through a needle ranging in diameter from 20 to 25 gauge.

22-23. (canceled)

24. The formulation of claim 2, wherein the formulation exhibits a storage cryostability comprising a period of at least two years at a temperature of 5° C. or the formulation exhibits stability at room temperature for up to four hours.

25-27. (canceled)

28. The formulation of claim 2, wherein the oligopeptide is further defined by the amino acid sequence selected from any one of SEQ ID NO: 2, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13.

29-36. (canceled)

37. The formulation of claim 2, wherein the oligopeptide is a free base form of the oligopeptide or an acid addition salt form of the oligopeptide.

38-40. (canceled)

41. A method of treating a subject identified as having or at risk of developing vascular dementia comprising administering to the subject the extended-release injectable gel formulation of claim 2.

42. The method of claim 41, wherein the formulation is administered to the subject:
(a) at a dosage of the oligopeptide of between 10-14 mg/day, 70 mg/week, 140 mg/two weeks, or 280 mg/month,
(b) once daily, once weekly, biweekly, bimonthly, or once monthly;
(c). through a needle having a diameter of 20 to 25 gauge; or
(d) by way of subcutaneous injection or intramuscular injection.

43-45. (canceled)

46. A method of preparing an injectable solution comprising an extended-release gel formulation comprising at least one biocompatible polymer comprising PLA or PLGA having dispersed therein an effective amount of an oligopeptide of SEQ ID NO: 1, the method comprising providing a first sterile syringe comprising a first solution comprising at least one biocompatible polymer comprising PLA or PLGA and a first solvent, providing a second sterile syringe comprising a second solution comprising an effective amount of an oligopeptide of SEQ ID NO: 1 and a second solvent, admixing the first solution and the second solution by joining the first syringe and the second syringe together and injecting the first solution in the first sterile syringe into the second syringe, wherein the admixing produces the extended-release gel formulation, and decoupling the first syringe and the second syringe.

47-65. (canceled)


SEQUENCE LISTING (13 sequences)

SEQ ID NO: 1 - protein, 8 aa, Artificial Sequence
  Synthetic Construct
  MISC_FEATURE (1)..(1): Xaa is Asp, Glu, or Ala
  MISC_FEATURE (2)..(2): Xaa is Arg, His, or Lys
  MISC_FEATURE (3)..(3): Xaa is Val, Ala, Ile, or Leu
  MISC_FEATURE (4)..(4): Xaa is Tyr, Phe, or Trp
  MISC_FEATURE (5)..(5): Xaa is Ile, Val, Ala, or Leu
  MISC_FEATURE (6)..(6): Xaa is His, Arg, or Lys
  MISC_FEATURE (7)..(7): Xaa is Pro, Gly, or Ser
  MISC_FEATURE (8)..(8): Xaa is Ser, Thr, hydroxyproline, or absent
XXXXXXXX

SEQ ID NO: 2 - protein, 7 aa, Artificial Sequence
  Synthetic Construct
DRVYIHP

SEQ ID NO: 3 - protein, 7 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
XXXXXXX

SEQ ID NO: 4 - protein, 7 aa, Artificial Sequence
  Synthetic Construct
DRVYIHP

SEQ ID NO: 5 - protein, 7 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
XXXXXXX

SEQ ID NO: 6 - protein, 7 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
XXXXXXX

SEQ ID NO: 7 - protein, 8 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
  MISC_FEATURE (8)..(8): Xaa is a D-unglycosylated serine residue
XXXXXXXX

SEQ ID NO: 8 - protein, 8 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
  MISC_FEATURE (8)..(8): Xaa is a D-glycosylated serine residue
XXXXXXXX

SEQ ID NO: 9 - protein, 8 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is D-Pro
  MISC_FEATURE (8)..(8): Xaa is a D-lactosylated serine residue
XXXXXXXX

SEQ ID NO: 10 - protein, 7 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa is D-Val
  MISC_FEATURE (4)..(4): Xaa is D-Tyr
  MISC_FEATURE (5)..(5): Xaa is D-Ile
  MISC_FEATURE (6)..(6): Xaa is D-His
  MISC_FEATURE (7)..(7): Xaa is a D-glycosylated serine residue
XXXXXXX

SEQ ID NO: 11 - protein, 8 aa, Artificial Sequence
  Synthetic Construct
  MISC_FEATURE (1)..(1): Xaa is D-Asp
  MISC_FEATURE (2)..(2): Xaa can be any D-amino acid residue
  MISC_FEATURE (3)..(3): Xaa can be any D-amino acid residue
  MISC_FEATURE (4)..(4): Xaa is D-Tyr
  MISC_FEATURE (5)..(5): Xaa is D-Ile
  MISC_FEATURE (6)..(6): Xaa can be any D-amino acid residue
  MISC_FEATURE (7)..(7): Xaa is D-Pro
  MISC_FEATURE (8)..(8): Xaa is a D-unglycosylated serine residue, a D-glycosylated serine residue, or a D-lactosylated serine residue
XXXXXXXX

SEQ ID NO: 12 - protein, 8 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa can be any D-amino acid residue
  MISC_FEATURE (4)..(4): Xaa is D-Tyr
  MISC_FEATURE (5)..(5): Xaa can be any D-amino acid residue
  MISC_FEATURE (6)..(6): Xaa is D-His
  MISC_FEATURE (7)..(7): Xaa is D-Pro
  MISC_FEATURE (8)..(8): Xaa is a D-unglycosylated serine residue, a D-glycosylated serine residue, or a D-lactosylated serine residue
XXXXXXXX

SEQ ID NO: 13 - protein, 8 aa, Artificial Sequence
  Synthetic Construct
  MISC_FEATURE (1)..(1): Xaa is D-Asp
  MISC_FEATURE (2)..(2): Xaa is D-Arg
  MISC_FEATURE (3)..(3): Xaa can be any D-amino acid residue
  MISC_FEATURE (4)..(4): Xaa can be any D-amino acid residue
  MISC_FEATURE (5)..(5): Xaa can be any D-amino acid residue
  MISC_FEATURE (6)..(6): Xaa is D-His
  MISC_FEATURE (7)..(7): Xaa is D-Pro
  MISC_FEATURE (8)..(8): Xaa is a D-unglycosylated serine residue, a D-glycosylated serine residue, or a D-lactosylated serine residue
XXXXXXXX